(19) United States
(12) Patent Application Publication
(10) Pub. No.: US 2025/0114196 A1
(43) Pub. Date: Apr. 10, 2025

(54) IMPLANT DELIVERY APPARATUS WITH A TELESCOPIC SHAFT

(71) Applicant: EDWARDS LIFESCIENCES CORPORATION, Irvine, CA (US)

(72) Inventor: Kurt Kelly Reed, Costa Mesa, CA (US)

(21) Appl. No.: 18/984,631

(22) Filed: Dec. 17, 2024

Related U.S. Application Data
(63) Continuation of application No. PCT/US2023/025832, filed on Jun. 21, 2023.
(60) Provisional application No. 63/366,897, filed on Jun. 23, 2022.

(51) Int. Cl. A61F 2/24

(52) U.S. Cl. CPC A61F 2/2427

(57) ABSTRACT

A delivery apparatus configured to deliver a prosthetic implant can include a handle, a first shaft extending through the handle, and a second shaft that is coaxial with the first shaft and surrounds at least a proximal end portion of the first shaft. The second shaft can include an outer shaft member positioned proximal to the handle and an inner shaft member that is axially movable relative to the outer shaft member. The second shaft can be movable between an axially extended configuration and an axially collapsed configuration. When the second shaft is in the axially extended configuration, the inner shaft member can extend into the handle. When the second shaft is in the axially collapsed configuration, the inner shaft member can be positioned proximal to the handle.

### CROSS-REFERENCE TO RELATED APPLICATIONS

This application is a continuation of PCT Patent Application No. PCT/US2023/025832, filed on Jun. 21, 2023, which application claims the benefit of U.S. Provisional Application No. 63/366,897, filed Jun. 23, 2022, each of these applications being incorporated by reference herein.

### FIELD

The present disclosure concerns examples of delivery apparatuses for delivering a prosthetic implant into a patient's body and associated handle assemblies.

### BACKGROUND

Prosthetic valves can be used to treat cardiac valvular disorders. Native heart valves (e.g., the aortic, pulmonary, tricuspid, and mitral valves) function to prevent backward flow or regurgitation, while allowing forward flow. These heart valves can be rendered less effective by congenital, inflammatory, infectious conditions, etc. Such conditions can eventually lead to serious cardiovascular compromise or death. For many years, the doctors attempted to treat such disorders with surgical repair or replacement of the valve during open heart surgery.

A transcatheter technique for introducing and implanting a prosthetic heart valve using a catheter in a manner that is less invasive than open heart surgery can reduce complications associated with open heart surgery. In this technique, a prosthetic valve can be mounted in a compressed state on the end portion of a catheter and advanced through a blood vessel of the patient until the valve reaches the implantation site. The valve at the catheter tip can then be expanded to its functional size at the site of the defective native valve, such as by inflating a balloon on which the valve is mounted or, for example, the valve can have a resilient, self-expanding stent or frame that expands the valve to its functional size when it is advanced from a delivery sheath at the distal end of the catheter. Optionally, the valve can have a balloon-expandable, self-expanding, mechanically expandable frame, and/or a frame expandable in multiple or a combination of ways.

In some instances, a transcatheter heart valve (THV) may be appropriately sized to be placed inside a particular native valve (e.g., a native aortic valve). As such, the THV may not be suitable for implantation at another native valve (e.g., a native mitral valve) and/or in a patient with a larger native valve. Additionally, or alternatively, the native tissue at the implantation site may not provide sufficient structure for the THV to be secured in place relative to the native tissue.

In certain instances, a docking device can be implanted first within the native valve and can be configured to receive a THV and secure (e.g., anchor) the THV in a desired position within the native valve. For example, the docking device can form a more circular and/or stable anchoring site at the native valve annulus in which a THV can be expanded and implanted. A transcatheter delivery apparatus can be used to deliver the docking device to the implantation site. In certain instances, the docking device can be arranged within the delivery apparatus, coaxial with additional components of the delivery apparatus.

Operating a delivery apparatus for implantation of a THV and/or a docking device involves complex steps and requires specialized skills. Accordingly, improvements to the transcatheter delivery apparatus to simplify its operation are desirable.

### SUMMARY

The present disclosure relates to devices and related methods for treating valvular regurgitation and/or other valve issues. Specifically, the present disclosure is directed to a delivery apparatus configured to deliver a prosthetic implant, such as a THV and/or a docking device, and the methods of implanting the prosthetic implant.

According to certain examples, a delivery apparatus configured to deliver a prosthetic implant can include a handle, a first shaft extending through the handle; and a second shaft that is coaxial with the first shaft and surrounds at least a proximal end portion of the first shaft. The second shaft can include an outer shaft member positioned proximal to the handle and an inner shaft member that is axially movable relative to the outer shaft member. The second shaft can be movable between an axially extended configuration and an axially collapsed configuration.

When the second shaft is in the axially extended configuration, the inner shaft member can extend into the handle. When the second shaft is in the axially collapsed configuration, the inner shaft member can be positioned proximal to the handle.

According to certain examples, a delivery apparatus configured to deliver a prosthetic implant can include a first shaft having a lumen and a first fluid port that is fluidly coupled to the lumen of the first shaft, and a second telescopic shaft having an outer shaft member fixedly mounted around a proximal portion of the first shaft and one or more inner shaft members that are coaxial with and axially movable relative to the first shaft. The one or more inner shaft members can include an inner-most shaft member. The first shaft can extend through a lumen of the inner-most shaft member. The second telescopic shaft can include a second fluid port that is fluidly coupled to the lumen of the inner-most shaft member.

According to certain examples, a delivery apparatus configured to deliver a prosthetic implant can include a first shaft, a second shaft having an outer shaft member fixedly mounted around a proximal portion of the first shaft and one or more nested inner shaft members that are configured to be axially movable relative to one another, and a sealing member configured to seal one or more annular spaces formed between the one or more inner shaft members and the outer shaft member. The one or more inner shaft members can include an inner-most shaft member. The first shaft can extend through the inner-most shaft member.

Certain aspects of the disclosure concern a system. The system can include a prosthetic implant and a delivery apparatus configured to deliver the prosthetic implant to a target implantation location. The delivery apparatus can include a pusher shaft positioned proximal to the prosthetic implant, a sleeve shaft having an outer shaft member fixedly mounted around a proximal portion of the pusher shaft and one or more inner shaft members that are coaxial with and axially movable relative to the pusher shaft, and a suture lock connected to a proximal end of the pusher shaft. The suture lock can be releasably connected to the prosthetic implant via a release suture extending through the pusher shaft. The one or more inner shaft members can include an inner-most shaft member. The pusher shaft can extend through the inner-most shaft member.

Certain examples of the disclosure also comprise a method. The method can include inserting a delivery apparatus loaded with a prosthetic device into a vasculature of a patient and deploying the prosthetic device at a target location within the vasculature of the patient. The delivery apparatus can include a pusher shaft positioned proximal to the prosthetic implant, a sleeve shaft having an outer shaft member fixedly mounted around a proximal portion of the pusher shaft and one or more inner shaft members that are coaxial with and axially movable relative to the pusher shaft, and a delivery shaft surrounding at least a distal portion of the pusher shaft and positioned distal to the outer shaft member. The one or more inner shaft members can include an inner-most shaft member. The pusher shaft can extend through the inner-most shaft member.

The above method can be performed on a living animal or on a simulation, such as on a cadaver, cadaver heart, anthropomorphic ghost, simulator (for example, with body parts, heart, tissue, etc. being simulated).

According to certain examples, a delivery apparatus configured to deliver a prosthetic implant can include a pusher shaft, a telescopic sleeve shaft having a fixed shaft segment and one or more movable shaft segments that are coaxial with the fixed shaft segment, and a dock sleeve connected to one of the one or more movable shaft segments and configured to cover the prosthetic implant. The fixed shaft segment can be fixedly mounted around a proximal portion of the pusher shaft. The one or more movable shaft segments can be axially movable relative to the pusher shaft.

In some examples, a delivery apparatus or a system comprises one or more of the components recited in Examples 1-90 and 108-115 described in the section “Additional Examples of the Disclosed Technology” below.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 schematically illustrates a first stage in an exemplary mitral valve replacement procedure where a guide catheter and a guidewire are inserted into a vasculature of a patient and navigated through the vasculature and into a heart of the patient, towards a native mitral valve of the heart.

FIG. 2A schematically illustrates a second stage in the exemplary mitral valve replacement procedure where a docking device delivery apparatus extending through the guide catheter is used to deploy a docking device at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is deploy a prosthetic heart valve within the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5 is a side perspective view of a docking device in a helical configuration, according to one example.

FIG. 5A is a cross-sectional view of the docking device of FIG. 5 taken along line 5A-5A, according to one example.

FIG. 5B is a cross-sectional view of the docking device taken along the same line as in FIG. 5A, except in FIG. 5B, the docking device is in a substantially straight delivery configuration.

FIG. 5C is a cross-sectional view of the docking device of FIG. 5 taken along line 5A-5A, according to another example.

FIG. 5D is a cross-sectional view of the docking device taken along the same line as in FIG. 5C, except in FIG. 5D, the docking device is in a substantially straight delivery configuration.

FIG. 6 is a perspective view of a prosthetic heart valve, according to one example.

FIG. 7 is a perspective view of a prosthetic heart valve, according to another example.

FIG. 8 schematically depicts a longitudinal cross-sectional view of a delivery apparatus including a pusher shaft and a telescopic sleeve shaft.

FIG. 9A schematically depicts a longitudinal cross-sectional view of a proximal portion of the delivery apparatus of FIG. 8, where the telescopic sleeve shaft is in an axially extended configuration.

FIG. 9B is a perspective view of a distal end portion of the delivery apparatus of FIG. 8, which illustrates an exemplary docking device being deployed from an outer shaft of the delivery apparatus and covered by a dock sleeve.

FIG. 10A schematically depicts a longitudinal cross-sectional view of a proximal portion of the delivery apparatus of FIG. 8, where the telescopic sleeve shaft is in a partially extended configuration.

FIG. 10B is a perspective view of a distal end portion of the delivery apparatus of FIG. 8, which illustrates the docking device of FIG. 9B being deployed from the outer shaft of the delivery apparatus with the dock sleeve partially removed from the docking device.

FIG. 11A schematically depicts a longitudinal cross-sectional view of a proximal portion of the delivery apparatus of FIG. 8, where the telescopic sleeve shaft is in an axially collapsed configuration.

FIG. 11B is a perspective view of a distal end portion of the delivery apparatus of FIG. 8, which illustrates the docking device of FIG. 9B being deployed from the outer shaft of the delivery apparatus with the dock sleeve completely removed from the docking device.

FIG. 12A schematically depicts a longitudinal cross-sectional view of a proximal portion of the delivery apparatus of FIG. 8, where two rollers are configured to move the telescopic sleeve shaft from an axially extended configuration to an axially collapsed configuration.

FIG. 12B schematically depicts a longitudinal cross-sectional view of a proximal portion of the delivery apparatus of FIG. 8, where one or more rollers are configured to move the telescopic sleeve shaft from an axially collapsed configuration to an axially extended configuration.

FIG. 13A schematically depicts a lead screw and a nut configured to actuate telescopic movement of the sleeve shaft of FIG. 8.

FIG. 13B schematically depicts a rack and a pinion configured to actuate telescopic movement of the sleeve shaft of FIG. 8.

FIG. 13C schematically depicts a rotatable cam configured to actuate telescopic movement of the sleeve shaft of FIG. 8.

FIG. 13D schematically depicts a rotatable drive shaft and a rod configured to actuate telescopic movement of the sleeve shaft of FIG. 8.

FIG. 14A schematically depicts two grippers configured to actuate telescopic movement of the sleeve shaft of FIG. 8.

FIG. 14B schematically depicts a slider configured to actuate telescopic movement of the sleeve shaft of FIG. 8.

FIG. 14C schematically depicts a biasing member configured to actuate telescopic movement of the sleeve shaft of FIG. 8.

FIG. 15A schematically depicts a side view of a telescopic sleeve shaft in a partially extended configuration, where the telescopic sleeve has an elastic sealing membrane, according to one example.

FIG. 15B schematically depicts a side view of the telescopic sleeve shaft of FIG. 15A in a fully extended configuration.

FIG. 15C schematically depicts a cross-sectional view of the telescopic sleeve shaft of FIG. 15A taken along an axis perpendicular to the longitudinal axis of the telescopic sleeve shaft.

FIG. 16A schematically depicts a side view of a telescopic sleeve shaft in a partially extended configuration, where the telescopic sleeve has a plurality of elastic sealing segments, according to one example.

FIG. 16B schematically depicts a side view of the telescopic sleeve shaft of FIG. 16A in a fully extended configuration.

FIG. 17 schematically depicts a longitudinal cross-sectional view of a telescopic sleeve shaft including a plurality of annular rings, according to one example.

### DETAILED DESCRIPTION

### General Considerations

It should be understood that the disclosed examples can be adapted to deliver and implant prosthetic devices in any of the native annuluses of the heart (e.g., the pulmonary, mitral, and tricuspid annuluses), and can be used with any of various delivery approaches (e.g., retrograde, antegrade, transseptal, transventricular, transatrial, etc.).

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (e.g., mechanically or chemically) coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

Directions and other relative references (e.g., inner, outer, upper, lower, etc.) may be used to facilitate discussion of the drawings and principles herein but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part, and the object remains the same. As used herein, “and/or” means “and” or “or,” as well as “and” and “or.”

As used in this application and in the claims, the term “shape-setting” can generally refer to a process of plastically deforming a material from a first configuration to a second configuration. The shape-setting process can result in the material becoming biased to the second configuration. In some examples, shape-setting can occur as a result of a physical stimulus, a chemical reaction, or a combination thereof.

In some examples, the shape-setting process can further impart shape-memory properties onto the material. As used in this application and in the claims, the term “shape-memory” can generally refer to the tendency of a material to elastically deform or return from a third configuration to the second configuration after the material is deformed from the second configuration to the third configuration. In some examples, the shape-memory property can be activated by an external stimulus, such as a temperature change, chemical reaction, or physical force.

As described herein, the term “inflow” can generally refer to a position, direction, or portion of the prosthetic heart valve that is closer an inlet into which blood flow enters the prosthetic heart valve. As described herein, the term “outflow” can generally refer to a position, direction, or portion of a prosthetic heart valve that is closer an outlet from which blood flow exits the prosthetic heart valve.

### Exemplary Transcatheter Heart Valve Replacement Procedure

Described herein are various systems, apparatuses, methods, or the like, that can be used in or with delivery apparatuses to deliver a prosthetic implant (e.g., a prosthetic valve, a docking device, etc.) into a patient body.

In certain examples, a delivery apparatus can be configured to deliver and implant a docking device at an implantation site, such as a native valve annulus. The docking device can be configured to more securely hold an expandable prosthetic valve implanted within the docking device, at the native valve annulus. For example, a docking device can provide or form a more circular and/or stable anchoring site, landing zone, or implantation zone at the implant site, in which a prosthetic valve can be expanded or otherwise implanted. By providing such anchoring or docking devices, replacement prosthetic valves can be more securely implanted and held at various valve annuluses, including at the mitral annulus which does not have a naturally circular cross-section.

In some examples, the docking device can be arranged within an outer shaft of the delivery apparatus. A sleeve shaft can cover or surround the docking device within the delivery apparatus and during delivery to a target implantation site. A pusher shaft can be disposed within the outer shaft, proximal to the docking device, and configured to push the docking device out of the outer shaft to position the docking device at the target implantation site. The sleeve shaft can also surround the pusher shaft within the outer shaft of the delivery apparatus. After positioning the docking device at the target implantation site, the sleeve shaft can be removed from the docking device and retracted back into the outer shaft of the delivery apparatus.

Fluid (e.g., a flush fluid, such as heparinized saline or the like) can be provided to a pusher shaft lumen defined within an interior of the pusher shaft, a delivery shaft lumen defined between the sleeve shaft and the outer shaft of the delivery apparatus, and a sleeve shaft lumen defined between the pusher shaft and the sleeve shaft. By providing a consistent flow of fluid through these lumens of the delivery apparatus, stagnation of blood within the delivery apparatus can be reduced or avoided, thereby reducing a risk of thrombus formation.

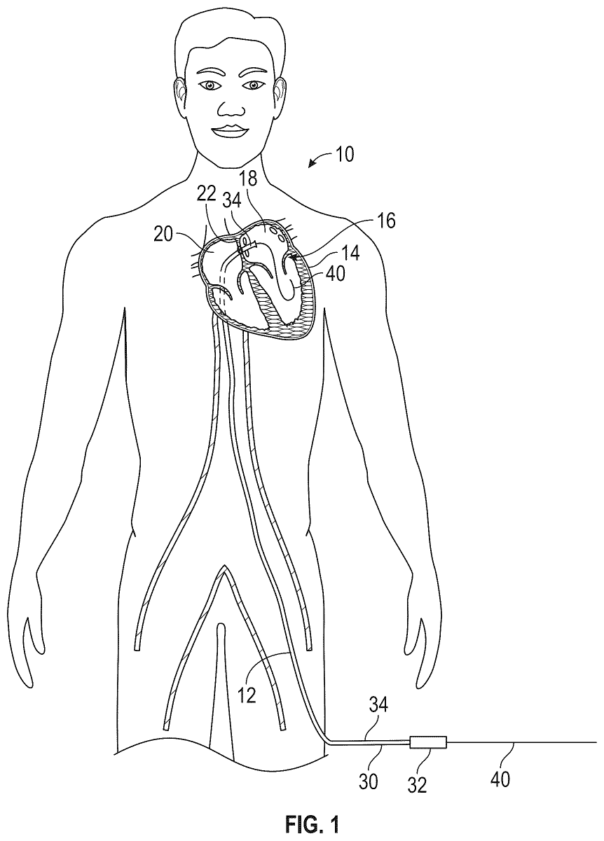

An exemplary transcatheter heart valve replacement procedure which utilizes a first delivery apparatus to deliver a docking device to a native valve annulus and then a second delivery apparatus to deliver a prosthetic heart valve (e.g., THV) inside the docking device is depicted in the schematic illustrations of FIGS. 1-4.

As introduced above, defective native heart valves may be replaced with THVs. However, in certain instances, such THVs may not be able to sufficiently secure themselves to the native tissue (e.g., to the leaflets and/or annulus of the native heart valve) and may undesirably shift around relative to the native tissue, leading to paravalvular leakage, valve malfunction, and/or other issues. Thus, a docking device may be implanted first at the native valve annulus and then the THV can be implanted within the docking device to help anchor the THV to the native tissue and provide a seal between the native tissue and the THV.

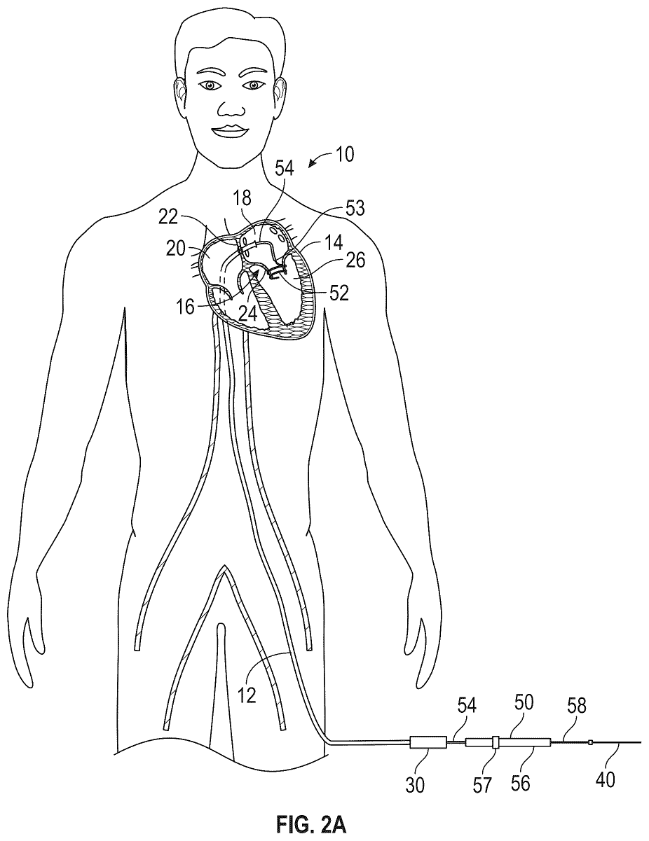

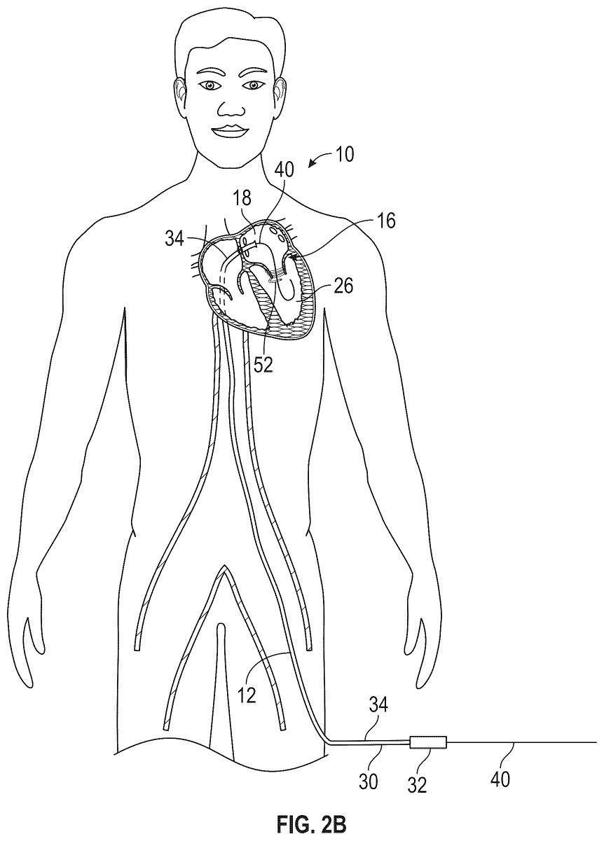

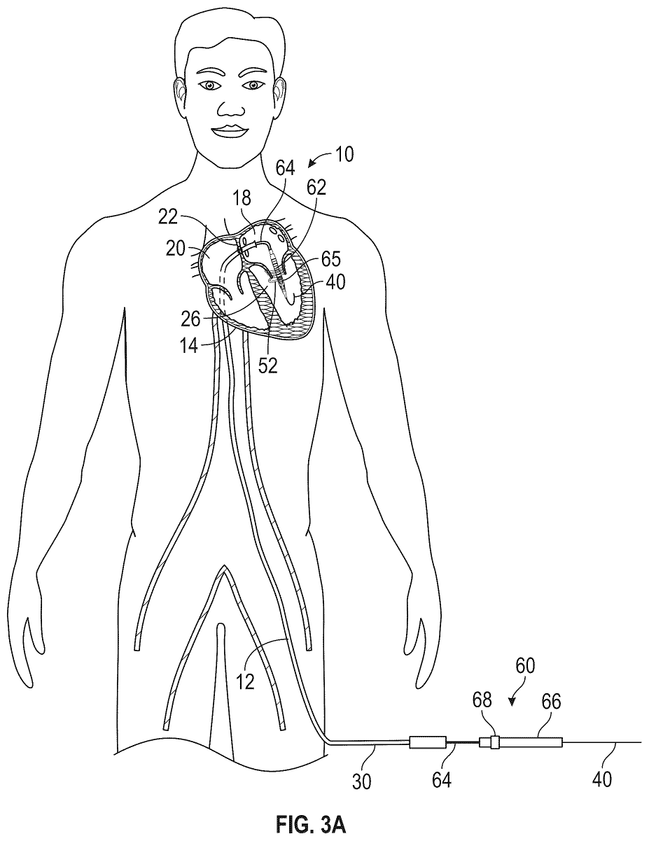

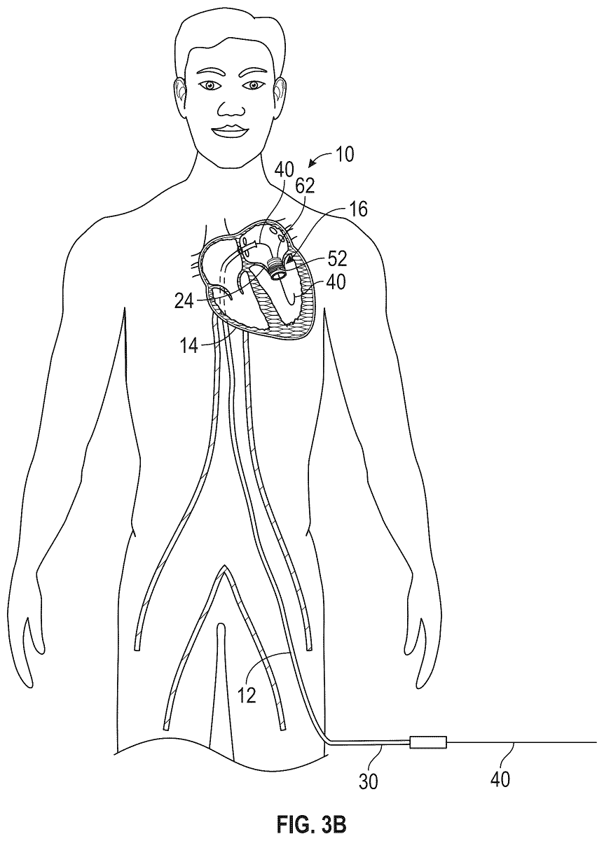

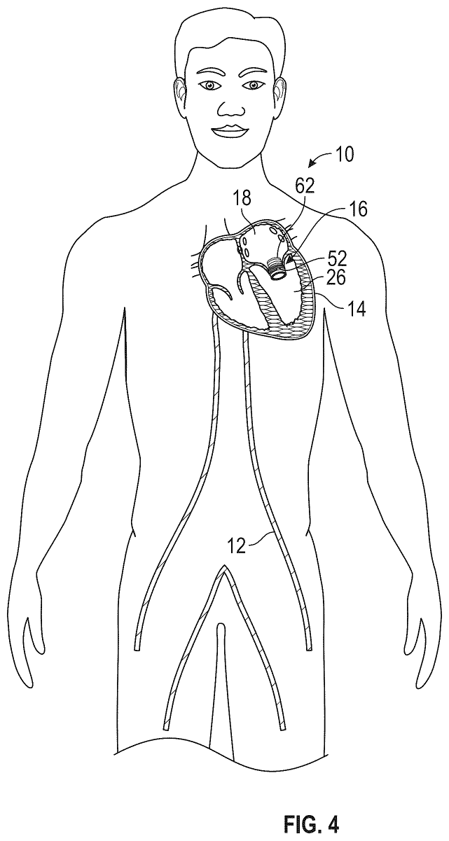

FIGS. 1-4 depict an exemplary transcatheter heart valve replacement procedure (e.g., a mitral valve replacement procedure) which utilizes a docking device 52 and a prosthetic heart valve 62, according to one example. During the procedure, a user can create a pathway to a patient's native heart valve using a guide catheter 30 (FIG. 1). The user can deliver and implant the docking device 52 at the patient's native heart valve using a docking device delivery apparatus 50 (FIG. 2A) and then removes the docking device delivery apparatus 50 from the patient 10 after implanting the docking device 52 (FIG. 2B). The user can then implant the prosthetic heart valve 62 within the implanted docking device 52 using a prosthetic valve delivery apparatus 60 (FIG. 3A). Thereafter, the user can remove the prosthetic valve delivery apparatus 60 from the patient 10 (FIG. 3B), as well as the guide catheter 30 (FIG. 4).

FIG. 1 depicts a first stage in a mitral valve replacement procedure, according to one example. As shown, the guide catheter 30 and a guidewire 40 can be inserted into a vasculature 12 of a patient 10 and navigated through the vasculature 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (e.g., the native mitral valve 16 or native mitral valve annulus).

Initially, the user may first make an incision in the patient's body to access the vasculature 12. For example, in the example illustrated in FIG. 1, the user may make an incision in the patient's groin to access a femoral vein. Thus, in such examples, the vasculature 12 may include a femoral vein.

After making the incision to access the vasculature 12, the user may insert the guide catheter 30, the guidewire 40, and/or additional devices (such as an introducer device or transseptal puncture device) through the incision and into the vasculature 12. The guide catheter 30 (which can also be referred to as an “introducer device,” “introducer,” or “guide sheath”) can be configured to facilitate the percutaneous introduction of various implant delivery devices (e.g., the docking device delivery apparatus 50 and the prosthetic valve delivery apparatus 60) into and through the vasculature 12 and may extend through the vasculature 12 and into the heart 14 but may stop short of the native mitral valve 16. The guide catheter 30 can comprise a handle 32 and a shaft 34 extending distally from the handle 32. The shaft 34 can extend through the vasculature 12 and into the heart 14 while the handle 32 can remain outside the body of the patient 10 and can be operated by the user in order to manipulate the shaft 34 (FIG. 1).

The guidewire 40 can be configured to guide the delivery apparatuses (e.g., the guide catheter 30, the docking device delivery apparatus 50, the prosthetic valve delivery apparatus 60, additional catheters, or the like) and their associated devices (e.g., docking device, prosthetic heart valve, and the like) to the implantation site within the heart 14, and thus may extend all the way through the vasculature 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 20. For example, after making the incision to access the vasculature 12, the user may insert a transseptal puncture device through the incision and into the vasculature 12. The user may guide the transseptal puncture device through the vasculature 12 and into the heart 14 (e.g., through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the vasculature 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the vasculature 12 and advance the guide catheter 30 into the left atrium 18 over the guidewire 40 (FIG. 1).

In some instances, an introducer device can be inserted through a lumen of the guide catheter 30 prior to inserting the guide catheter 30 into the vasculature 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 can be implanted at the native mitral valve 16 of the heart 14 of the patient 10 using a docking device delivery apparatus 50 (which may also be referred to as an “implant catheter,” or a “docking device delivery device,” or simply “delivery apparatus”).

In general, the docking device delivery apparatus 50 can include a delivery shaft 54 (which may also be referred to as an “outer shaft”), a handle 56, and a pusher assembly 58. The delivery shaft 54 can be configured to be advanced through the patient's vasculature 12 and to the implantation site (e.g., native mitral valve 16) by the user, and may be configured to retain the docking device 52 in a distal end portion 53 of the delivery shaft 54. In some examples, the distal end portion 53 of the delivery shaft 54 can retain the docking device 52 therein in a substantially straightened delivery configuration.

The handle 56 of the docking device delivery apparatus 50 can be configured to be gripped and/or otherwise held by the user to advance the delivery shaft 54 through the patient's vasculature 12. Specifically, the handle 56 can be coupled to a proximal end of the delivery shaft 54 and can be configured to remain accessible to the user (e.g., outside the body of the patient 10) during the docking device implantation procedure. In this way, the user can advance the delivery shaft 54 through the patient's vasculature 12 by exerting a force on (e.g., pushing) the handle 56. In some examples, the delivery shaft 54 can be configured to carry the pusher assembly 58 and/or the docking device 52 with it as it advances through the patient's vasculature 12. In this way, the docking device 52 and/or the pusher assembly 58 can advance through the patient's vasculature 12 in lockstep with the delivery shaft 54 as the user grips the handle 56 and pushes the delivery shaft 54 deeper into the patient's vasculature 12.

In some examples, the handle 56 can comprise one or more articulation members 57 that are configured to aid in navigating the delivery shaft 54 through the vasculature 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the vasculature 12 and/or within the heart 14.

The pusher assembly 58 can be configured to deploy and/or implant the docking device 52 at the implantation site (e.g., the native mitral valve 16). For example, the pusher assembly 58 can be configured to be adjusted by the user to push the docking device 52 out of the distal end portion 53 of the delivery shaft 54. A pusher shaft of the pusher assembly 58 can extend through the delivery shaft 54 and can be disposed adjacent to the docking device 52 within the delivery shaft 54. In some examples, the docking device 52 can be releasably coupled to the pusher shaft of the pusher assembly 58 via a connection mechanism of the docking device delivery apparatus 50 such that the docking device 52 can be released after being deployed at the native mitral valve 16. Because the docking device 52 is retained by, held, and/or otherwise coupled to the pusher assembly 58, the docking device 52 can advance in lockstep with the pusher assembly 58 through and/or out of the delivery shaft 54.

In addition to the pusher shaft, in certain instances, the pusher assembly 58 can also include a sleeve shaft. The pusher shaft can be configured to advance the docking device 52 through the delivery shaft 54 and out of the distal end portion 53 of the delivery shaft 54, while the sleeve shaft, when included, can have a distal dock sleeve configured to cover the docking device 52 within the delivery shaft 54 and while pushing the docking device 52 out of the delivery shaft 54 and positioning the docking device 52 at the implantation site. In some examples, the pusher shaft can be covered, at least in part, by the sleeve shaft. In certain examples, the sleeve shaft can include telescopic shaft members, as described further below.

In some examples, the pusher assembly 58 can comprise a pusher handle that is coupled to the pusher shaft and that is configured to be gripped and pushed by the user to translate the pusher shaft axially relative to the delivery shaft 54 (e.g., to push the pusher shaft into and/or out of the distal end portion 53 of the delivery shaft 54). The dock sleeve can be configured to be retracted and/or withdrawn from the docking device 52, after positioning the docking device 52 at the target implantation site. For example, the pusher assembly 58 can include a sleeve handle that is coupled to the sleeve shaft and is configured to be pulled by a user to retract (e.g., axially move) the sleeve shaft relative to the pusher shaft, thereby retracting the dock sleeve.

The pusher assembly 58 can be removably coupled to the docking device 52, and as such can be configured to release, detach, decouple, and/or otherwise disconnect from the docking device 52 once the docking device 52 has been deployed at the target implantation site. As just one example, the pusher assembly 58 may be removably coupled to the docking device 52 via a thread, string, yarn, suture, or other suitable material that is tied or sutured to the docking device 52.

In some examples, the pusher assembly 58 can include a suture lock assembly (also referred to as a “suture lock”) that is configured to receive and/or hold the thread or other suitable material that is coupled to the docking device 52 via a suture. The thread or other suitable material that forms the suture can extend from the docking device 52, through the pusher assembly 58, to the suture lock assembly. The suture lock assembly can also be configured to cut the suture to release, detach, decouple, and/or otherwise disconnect the docking device 52 from the pusher assembly 58. For example, the suture lock assembly can comprise a cutting mechanism that is configured to be adjusted by the user to cut the suture.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the vasculature 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the vasculature 12 and the heart 14, the user may adjust the one or more articulation members 57 of the handle 56 to navigate the various turns, corners, constrictions, and/or other obstacles in the vasculature 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may then deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16. For example, the user can maintain the position of the pusher assembly 58 (e.g., by exerting a holding and/or pushing force on the pusher shaft) while retracting the delivery shaft 54 proximally so that the delivery shaft 54 withdraws and/or otherwise retracts relative to the docking device 52 and the pusher assembly 58. In this way, the pusher assembly 58 can hold the docking device 52 in place while the user retracts the delivery shaft 54, thereby releasing the docking device 52 from the delivery shaft 54. In some examples, the user can also remove the dock sleeve from the docking device 52, e.g., by retracting the sleeve shaft.

After deploying and implanting the docking device 52 at the native mitral valve 16, the user may disconnect the docking device delivery apparatus 50 from the docking device 52. Once the docking device 52 can be disconnected from the docking device delivery apparatus 50 (e.g., by cutting the suture tied to the docking device 52), the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts a third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 can have a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 within the docking device 52 using a prosthetic valve delivery apparatus 60.

As shown in FIG. 3A, the prosthetic valve delivery apparatus 60 can comprise a delivery shaft 64 and a handle 66. The delivery shaft 64 can extend distally from the handle 66. The delivery shaft 64 can be configured to extend into the patient's vasculature 12 to deliver, implant, expand, and/or otherwise deploy the prosthetic heart valve 62 within the docking device 52 at the native mitral valve 16. The handle 66 can be configured to be gripped and/or otherwise held by the user to advance the delivery shaft 64 through the patient's vasculature 12.

In some examples, the handle 66 can comprise one or more articulation members 68 that are configured to aid in navigating the delivery shaft 64 through the vasculature 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the vasculature 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 can be mounted around the expansion mechanism 65 (e.g., the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (e.g., the delivery shaft 64) into the patient 10 through the guide catheter 30 and over the guidewire 40. The user can continue to advance the prosthetic valve delivery apparatus 60 along the guidewire 40 (e.g., through the vasculature 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the vasculature 12 and the heart 14, the user can adjust the one or more articulation members 68 of the handle 66 to navigate the various turns, corners, constrictions, and/or other obstacles in the vasculature 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52. In some examples, the user can lock the prosthetic heart valve 62 in its fully expanded position (e.g., with a locking mechanism) to prevent the prosthetic heart valve 62 from collapsing.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 can be received and retained within the docking device 52.

As also shown in FIG. 3B, after the prosthetic heart valve 62 has been fully deployed and implanted within the docking device 52 at the native mitral valve 16, the prosthetic valve delivery apparatus 60 (including the delivery shaft 64) can be removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10. The docking device 52 can be configured to provide a seal between the prosthetic heart valve 62 and the leaflets 24 of the native mitral valve 16 to reduce paravalvular leakage around the prosthetic heart valve 62. Specifically, the docking device 52 can initially constrict the leaflets 24 of the native mitral valve 16. The prosthetic heart valve 62 can then push the leaflets 24 against the docking device 52 as it radially expands within the docking device 52. Thus, the docking device 52 and the prosthetic heart valve 62 can be configured to sandwich the leaflets 24 of the native mitral valve 16 when the prosthetic heart valve 62 is expanded within the docking device 52. In this way, the docking device 52 can provide a seal between the leaflets 24 of the native mitral valve 16 and the prosthetic heart valve 62 to reduce paravalvular leakage around the prosthetic heart valve 62.

In some examples, one or more of the docking device delivery apparatus 50, the prosthetic valve delivery apparatus 60, and/or the guide catheter 30 can comprise one or more fluid ports that are configured to supply flushing fluid to the lumens thereof to prevent and/or reduce the likelihood of blood clot (e.g., thrombus) formation. Example fluid ports that can be used to inject flushing fluid into a docking device delivery apparatus are described further below.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned or disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

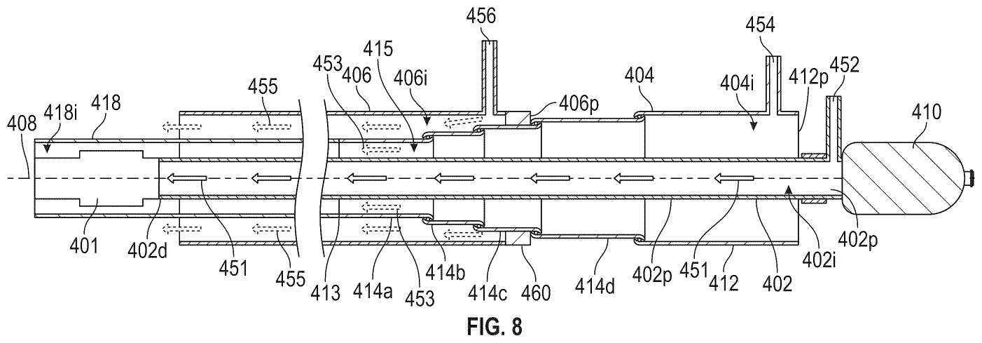

Further details of the docking device delivery apparatus are described further below with reference to FIGS. 8-17.

Additional examples of the docking device delivery apparatus, including its variants, and methods of implanting a docking device and implanting a prosthetic valve within the docking device are described in International Patent Applications No. PCT/US2020/36577 and PCT/US2021/056150, and U.S. Patent Publication Nos. US2018/0318079, US2018/0263764, and US2018/0177594, which are all incorporated by reference herein in their entireties.

### Exemplary Docking Device

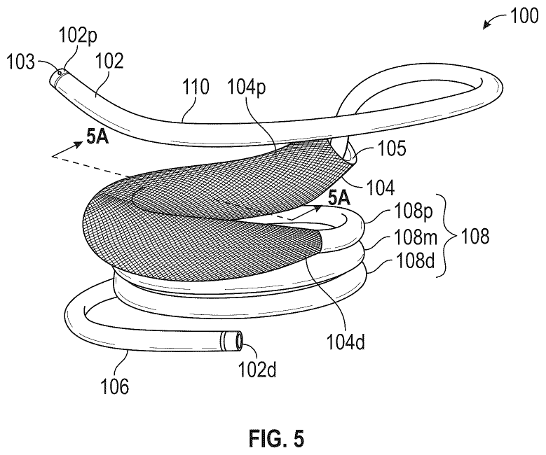

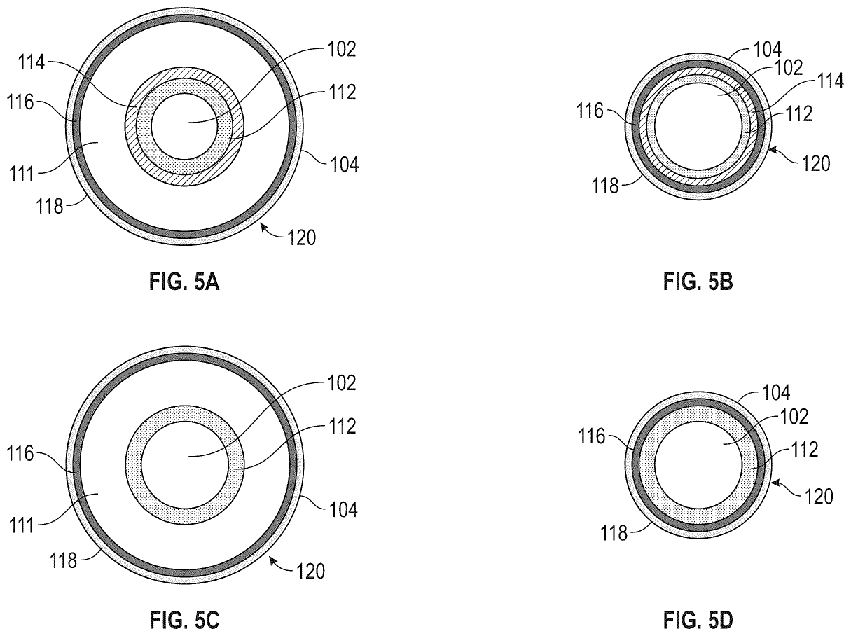

FIG. 5 shows an example of a docking device 100 configured to receive a prosthetic heart valve. For example, the docking device 100 can be implanted within a native valve annulus, as described above with reference to FIGS. 2-4. As depicted in FIGS. 2A-2B and 3-4, the docking device 100 can be configured to receive and secure a prosthetic valve within the docking device, thereby securing the prosthetic valve at the native valve annulus.

Referring to FIG. 5, the docking device 100 can comprise two main components: a coil 102 and a guard member 104 covering at least a portion of the coil 102. In certain examples, the coil 102 can include a shape memory material (e.g., Nitinol) such that the docking device 100 (and the coil 102) can move from a substantially straight configuration (also referred to as “delivery configuration”) when disposed within a delivery sheath of a delivery apparatus (as described more fully below) to a helical configuration (also referred to as “deployed configuration,” as shown in FIG. 5) after being removed from the delivery sheath.

The coil 102 has a proximal end 102p and a distal end 102d. When being disposed within the delivery sheath (e.g., during delivery of the docking device into the vasculature of a patient), a body of the coil 102 between the proximal end 102p and distal end 102d can form a generally straight delivery configuration (e.g., without any coiled or looped portions) so as to maintain a small radial profile when moving through the patient's vasculature. After being removed from the delivery sheath and deployed at an implant position, the coil 102 can move from the delivery configuration to the helical deployed configuration and wrap around native tissue adjacent the implant position. For example, when implanting the docking device at the location of a native valve, the coil 102 can be configured to surround native leaflets of the native valve (and the chordae tendineae that connects native leaflets to adjacent papillary muscles, if present).

The docking device 100 can be releasably coupled to a delivery apparatus. For example, in certain examples, the docking device 100 can be coupled to a delivery apparatus via a release suture that can be tied to the docking device 100 and cut for removal (as described further below with reference to FIG. 11B). In one example, the release suture can be tied to the docking device 100 through an eyelet or eyehole 103 located adjacent to the proximal end 102p of the coil. In another example, the release suture can be tied around a circumferential recess that is located adjacent to the proximal end 102p of the coil 102.

In some examples, the docking device 100 in the deployed configuration can be configured to fit at the mitral valve position. In other examples, the docking device 100 can also be shaped and/or adapted for implantation at other native valve positions as well, such as at the tricuspid valve. In some examples, the geometry of the docking device 100 can be configured to engage the native anatomy, which can, for example, provide for increased stability and reduction of relative motion between the docking device 100, the prosthetic valve docked therein, and/or the native anatomy. Reduction of such relative motion can, among other things, prevent material degradation of components of the docking device 100 and/or the prosthetic valve docked therein and/or prevent damage or trauma to the native tissue.

As shown in FIG. 5, the coil 102 in the deployed configuration can include a leading turn 106 (or “leading coil”), a central region 108, and stabilization turn 110 (or “stabilization coil”). The central region 108 can possess one or more helical turns having substantially equal inner diameters. The leading turn 106 can extend from a distal end of the central region 108 and has a diameter greater than the diameter of the central region 108 (in one or more configurations). The stabilization turn 110 can extend from a proximal end of the central region 108 and has a diameter greater than the diameter of the central region 108 (in one or more configurations).

In certain examples, the central region 108 can include a plurality of helical turns, such as a proximal turn 108p in connection with the stabilization turn 110, a distal turn 108d in connection with the leading turn 106, and one or more intermediate turns 108m disposed between the proximal turn 108p and the distal turn 108d. In the example shown in FIG. 5, there is only one intermediate turn 108m between the proximal turn 108p and the distal turn 108d. In other examples, there are more than one intermediate turns 108m between the proximal turn 108p and the distal turn 108d. Some of the helical turns in the central region 108 can be full turns (i.e., rotating 360 degrees). In some examples, the proximal turn 108p and/or the distal turn 108d can be partial turns (e.g., rotating less than 360 degrees, such as 180 degrees, 270 degrees, etc.).

The size of the docking device 100 can be generally selected based on the size of the desired prosthetic valve to be implanted into the patient. In certain examples, the central region 108 can be configured to retain a radially expandable prosthetic valve. For example, the inner diameter of the helical turns in the central region 108 can be configured to be smaller than an outer diameter of the prosthetic valve when the prosthetic valve is radially expanded so that additional radial tension can act between the central region 108 and the prosthetic valve to hold the prosthetic valve in place. The helical turns (e.g., 108p, 108m, 108d) in the central region 108 are also referred to herein as “functional turns.”

The stabilization turn 110 can be configured to help stabilize the docking device 100 in the desired position. For example, the radial dimension of the stabilization turn 110 can be significantly larger than the radial dimension of the coil in the central region 108, so that the stabilization turn 110 can flare or extend sufficiently outwardly so as to abut or push against the walls of the circulatory system, thereby improving the ability of the docking device 100 to stay in its desired position prior to the implantation of the prosthetic valve. In some examples, the diameter of stabilization turn 110 is desirably larger than the annulus, native valve plane, and atrium for better stabilization. In some examples, the stabilization turn 110 can be a full turn (i.e., rotating about 360 degrees). In some examples, the stabilization turn 110 can be a partial turn (e.g., rotating between about 180 degrees and about 270 degrees).

In one particular example, when implanting the docking device 100 at the native mitral valve location, the functional turns in the central region 108 can be disposed substantially in the left ventricle and the stabilization turn 110 can be disposed substantially in the left atrium. The stabilization turn 110 can be configured to provide one or more points or regions of contact between the docking device 100 and the left atrial wall, such as at least three points of contact in the left atrium or complete contact on the left atrial wall. In certain examples, the points of contact between the docking device 100 and the left atrial wall can form a plane that is approximately parallel to a plane of the native mitral valve.

As noted above, the leading turn 106 can have a larger radial dimension than the helical turns in the central region 108. The leading turn 106 can help more easily guide the coil 102 around and/or through the chordae tendineae geometry and adequately around all native leaflets of the native valve (e.g., the native mitral valve, tricuspid valve, etc.). For example, once the leading turn 106 is navigated around the desired native anatomy, the remaining coil (such as the functional turns) of the docking device 100 can also be guided around the same features. In some examples, the leading turn 106 can be a full turn (i.e., rotating about 360 degrees). In some examples, the leading turn 106 can be a partial turn (e.g., rotating between about 180 degrees and about 270 degrees). In some examples, when a prosthetic valve is radially expanded within the central region 108 of the coil, the functional turns in the central region 108 can be further radially expanded. As a result, the leading turn 106 can be pulled in the proximal direction and become a part of the functional turns in the central region 108.

As shown in FIGS. 5A-5D, in certain examples, at least a portion of the coil 102 can be surrounded by a first cover 112. In certain examples, the first cover 112 can cover an entire length of the coil 102. In certain examples, the first cover 112 covers only selected portion(s) of the coil 102. In certain examples, the first cover 112 can be coated on and/or bonded on the coil 102. In certain examples, the first cover 112 can be a cushioned, padded-type layer protecting the coil. The first cover 112 can be constructed of various native and/or synthetic materials. In one particular example, the first cover 112 can include expanded polytetrafluoroethylene (ePTFE). In certain examples, the first cover 112 can be fixedly attached to the coil 102 (e.g., by means of textured surface resistance, suture, glue, thermal bonding, or any other means) so that relative axial movement between the first cover 112 and the coil 102 is restricted or prohibited.

As shown in FIGS. 5A-5B, in some examples, the docking device 100 can also include a retention element 114 surrounding at least a portion of the coil 102 and at least being partially covered by the guard member 104. In some instances, the retention element 114 can comprise a braided material. In addition, the retention element 114 can provide a surface area that encourages or promotes tissue ingrowth and/or adherence, and/or reduce trauma to native tissue. For example, the retention element 114 can have a textured outer surface configured to promote tissue ingrowth. In certain instances, the retention element 114 can be impregnated with growth factors to stimulate or promote tissue ingrowth.

In certain examples, at least a portion of the first cover 112 can be surrounded by the retention element 114. In some examples, the first cover 112 can extend through an entire length of the retention element 114. In some examples, a distal end portion of the retention element 114 can extent axially beyond (i.e., positioned distal to) the distal end of the guard member 104, and a proximal end portion of the retention element 114 can extend axially beyond (i.e., positioned proximal to) a proximal end 105 of the guard member 104 to aid retention of prosthetic valve and tissue ingrowth.

The retention element 114 can be designed to interact with the guard member 104 to limit or resist motion of the guard member 104 relative to the coil 102. For example, the proximal end 105 of the guard member 104 can have an inner diameter that is about the same as an outer diameter of the retention element 114. As such, an inner surface of the guard member 104 at the proximal end 105 can frictionally interact or engage with the retention element 114 so that axial movement of the proximal end 105 of the guard member 104 relative to the coil 102 can be impeded by a frictional force exerted by the retention element 114.

As depicted in FIGS. 5A-5D, in certain examples, the guard member 104 can include an expandable member 116 and a second cover 118 surrounding an outer surface of the expandable member 116. In certain examples, the expandable member 116 surrounds at least a portion of the first cover 112. In certain examples, the first cover 112 can extend (completely or partially) through the expandable member 116.

In certain examples, the expandable member 116 can include a braided structure, such as a braided wire mesh or lattice. In certain examples, the expandable member 116 can include a shape memory material that is shape set and/or pre-configured to expand to a particular shape and/or size when unconstrained (e.g., when deployed at a native valve location). For example, the expandable member 116 can have a braided structure containing a shape memory alloy with Superelastic properties, such as Nitinol. In certain examples, the expandable member 116 can have a braided structure containing a ternary shape memory alloy with Superelastic properties, such as NiTiX where X can be chromium (Cr), cobalt (Co), zirconium (Zr), hafnium (Hf), etc. In certain examples, the expandable member 116 can comprise a metallic material that does not have the shape memory properties. Examples of such metallic material include cobalt-chromium, stainless steel, etc. In one specific example, the expandable member 116 can comprise nick-free austenitic stainless steel in which nickel can be completely replaced by nitrogen. In another specific example, the expandable member 116 can comprise cobalt-chromium or cobalt-nickel-chromium-molybdenum alloy with significantly low density of titanium. The number of wires (or fibers, strands, or the like) forming the braided structure can be selected to achieve a desired elasticity and/or strength of the expandable member 116. In certain examples, the number of wires used to braid the expanding member 116 can range from 16 to 128 (e.g., 32 wires, 48 wires, 64 wires, 96 wires, etc.). In certain examples, the braid density can range from 20 picks per inch (PPI) to 70 PPI, or from 25 PPI to 65 PPI. In one specific example, the braid density is about 36 PPI. In another specific example, the braid density is about 40 PPI. In certain examples, the diameter of the wires can range from about 0.002 inch to about 0.004 inch. In one particularly example, the diameter of the wires can be about 0.003 inch. In another example, the expandable member 116 can be a combination of braided wire (which can include a shape memory material or non-shape memory material) and a polymeric material and/or textile (e.g., polyethylene terephthalate (PET), polytetrafluoroethylene (PTFE), polyether ether ketone (PEEK), thermoplastic polyurethane (TPU), etc.). For example, the expandable member 116 can include a braided wireframe embedded in a polymeric material.

In some examples, the expandable member 116 can include a braided metallic wireframe coated with an elastomer (e.g., ePTFE, TPU, or the like), which can elastically deform as the braided wireframe expands and/or compresses. In some examples, the expandable member 116 can comprise a braid and/or weave that includes one or more metallic wires and one or more polymeric fibers. In other words, the metallic wires and the polymeric fibers can be interwoven together to define a braided structure. In some instances, the polymeric fibers can have the same or about the same diameter as the metallic wires. In other instances, the polymeric fibers can have a smaller diameter (e.g., microfibers) than the metallic wires, or vice versa.

In yet another example, the expandable member 116 can include a polymeric material, such as a thermoplastic material (e.g., PET, polyether ether ketone (PEEK), thermoplastic polyurethane (TPU), etc.), without a braided wireframe.

In certain examples, the expandable member 116 can include a foam structure. For example, the expandable member can include an expandable memory foam which can expand to a specific shape or specific pre-set shape upon removal of a crimping pressure (e.g., removal of the docking device 100 from the delivery sheath) prior to delivery of the docking device.

The expandable member 116 can extend radially outwardly from the coil 102 and is movable between a radially compressed (and axially elongated) state and a radially expanded (and axially foreshortened) state. That is, the expandable member 116 can axially foreshorten when it moves from the radially compressed state to the radially expanded state and can axially elongate when it moves from the radially expanded state to the radially compressed state.

The second cover 118 can be configured to be so elastic that when the expandable member 116 moves from the radially compressed (and axially elongated) state to the radially expanded (and axially foreshortened) state, the second cover 118 can also radially expand and axially foreshorten together with the expandable member 116. In other words, the guard member 104, as a whole, can move from a radially compressed (and axially elongated) state to a radially expanded (and axially foreshortened) state.

In certain examples, the second cover 118 can be configured to be atraumatic to native tissue and/or promote tissue ingrowth into the second cover 118. For example, the second cover 118 can have pores to encourage tissue ingrowth. In another example, the second cover 118 can be impregnated with growth factors to stimulate or promote tissue ingrowth. The second cover 118 can be constructed of any suitable material, including foam, cloth, fabric, and/or polymer, which is flexible to allow for compression and expansion of the second cover. In one example, the second cover 118 can include a fabric layer constructed from a thermoplastic polymer material, such as polyethylene terephthalate (PET).

The guard member 104 can constitute a part of a cover assembly for the docking device 100. In some examples, the cover assembly can also include the first cover 112. In some examples, the cover assembly can further include the retention element 114.

In certain examples, when the docking device 100 is in the deployed configuration, the guard member 104 can be configured to cover a portion of the stabilization turn 110 of the coil 102. In certain examples, the guard member 104 can be configured to cover at least a portion of the central region 108 of the coil 102, such as a portion of the proximal turn 108p. In certain examples, the guard member 104 can extend over the entirety of the coil 102.

In some examples, the guard member 104 can radially expand so as to help prevent and/or reduce paravalvular leakage. Specifically, the guard member 104 can be configured to radially expand such that an improved seal is formed closer to and/or against the prosthetic valve deployed within the docking device 100. In some examples, the guard member 104 can be configured to prevent and/or inhibit leakage at the location where the docking device 100 crosses between leaflets of the native valve (e.g., at the commissures of the native leaflets). For example, without the guard member 104, the docking device 100 may push the native leaflets apart at the point of crossing the native leaflets and allow for leakage at that point (e.g., along the docking device or to its sides). However, the guard member 104 can be configured to expand to cover and/or fill any opening at that point and inhibit leakage along the docking device 100.

In certain examples, when the docking device 100 is deployed at a native atrioventricular valve, the guard member 104 can cover predominantly a portion of the stabilization turn 110 and/or a portion of the central region 108. As such, the guard member 104 can help covering an atrial side of the atrioventricular valve to prevent and/or inhibit blood from leaking through the native leaflets, commissures, and/or around an outside of the prosthetic valve by blocking blood in the atrium from flowing in an atrial to ventricular direction (i.e., antegrade blood flow)-other than through the prosthetic valve. Positioning the guard member 104 on the atrial side of the valve can additionally or alternatively help reduce blood in the ventricle from flowing in a ventricular to atrial direction (i.e., retrograde blood flow).

In some examples, the guard member 104 can be positioned on a ventricular side of an atrioventricular valve to prevent and/or inhibit blood from leaking through the native leaflets, commissures, and/or around an outside of the prosthetic valve by blocking blood in the ventricle from flowing in a ventricular to atrial direction (i.e., retrograde blood flow). Positioning the guard member 104 on the ventricular side of the valve can additionally or alternatively help reduce blood in the atrium from flowing in the atrial direction to ventricular direction (i.e., antegrade blood flow)—other than through the prosthetic valve.

In some examples, a distal end portion 104d of the guard member 104 can be fixedly coupled to the coil 102 (e.g., via a suture), and a proximal end portion 104p of the guard member 104 can be axially moveable relative to the coil 102.

When the docking device 100 is retained within the delivery sheath in the substantially straight configuration, the expandable member 116 can be radially compressed by the delivery sheath and remain in the radially compressed (and axially elongated) state. The radially compressed (and axially elongated) expandable member 116 can contact the retention element 114 (see, e.g., FIG. 5B) or the first cover 112 (see, e.g., FIG. 5D) so that no gap or cavity exists between the retention element 114 and the expandable member 116 or between the first cover 112 (and/or the coil 102) and the expandable member 116.

After the docking device 100 is removed from the delivery sheath and changes from the delivery configuration to the deployed configuration, the guard member 104 can also move from a delivery configuration to a deployed configuration. In certain examples, a dock sleeve can be configured to cover and retain the docking device 100 within the delivery sheath when navigating the delivery sheath through the patient's native valve. The dock sleeve can also, for example, help to guide the docking device 100 around the native leaflets and chordae. Retraction of the dock sleeve relative to the docking device 100 can expose the guard member 104 and cause it to move from the delivery configuration to the deployed configuration. Specifically, without the constraint of the delivery sheath and the dock sleeve, the expandable member 116 can radially expand (and axially foreshorten) so that a gap or cavity 111 can be created between the retention element 114 and the expandable member 116 (see, e.g., FIG. 5A) and/or between the first cover 112 and the expandable member 116 (see, e.g., FIG. 5C).

Because the distal end portion 104d of the guard member 104 is fixedly coupled to the coil 102 and the proximal end portion 104p of the guard member 104 can be axially moveable relative to the coil 102, the proximal end portion 104p of the guard member 104 can slide axially over the first cover 112 and toward the distal end 102d of the coil 102 when the expandable member 116 moves from the radially compressed state to the radially expanded state. As a result, the proximal end portion 104p of the guard member 104 can be disposed closer to the proximal end 102p of the coil 102 when the expandable member 116 is in the radially compressed state than in the radially expanded state.

In certain examples, the second cover 118 can be configured to engage with the prosthetic valve deployed within the docking device 100 so as to form a seal and reduce paravalvular leakage between the prosthetic valve and the docking device 100 when the expandable member 116 is in the radially expanded state. The second cover 118 can also be configured to engage with the native tissue (e.g., the native annulus and/or native leaflets) to reduce paravalvular leakage between the docking device and/or the prosthetic valve and the native tissue.

In certain examples, when the guard member 104 is in the radially expanded state, the proximal end portion 104p of the guard member 104 can have a tapered shape, such that the diameter of the proximal end portion 104p gradually increases from the proximal end 105 of the guard member 104 to a distally located body portion of the guard member 104. This can, for example, help to facilitate loading the docking device into a delivery sheath of the delivery apparatus and/or retrieval and/or re-positioning of the docking device into the delivery apparatus during an implantation procedure. In addition, due to its small diameter, the proximal end 105 of the guard member 104 can frictionally engage with the retention element 114 so that the retention element 114 can reduce or prevent axial movement of the proximal end portion 104p of the guard member 104 relative to the coil 102.

Additional examples of the docking device and the guard member, as well as methods of assembling the docking device (e.g., attaching the guard member to the coil) are described in International Patent Application PCT/US2021/056150.

### Exemplary Prosthetic Valves

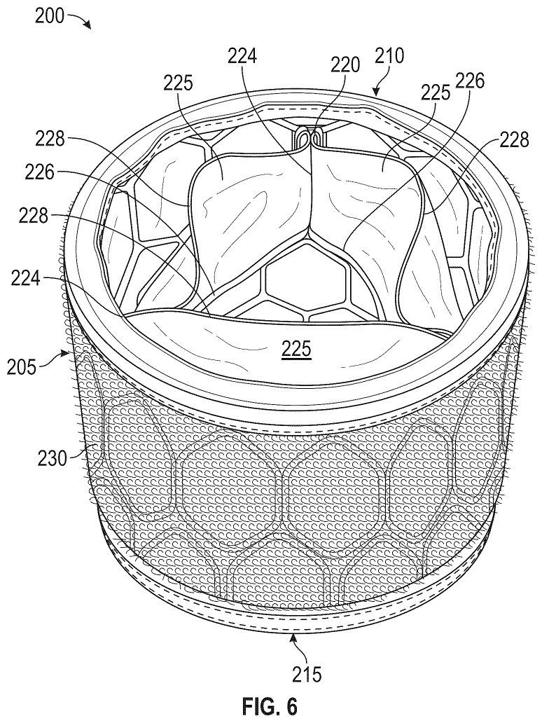

FIG. 6 illustrates a perspective view of a prosthetic heart valve 200, according to one example. In certain examples, the prosthetic heart valve 200 can be a dry bioprosthetic heart valve.

As shown, the prosthetic heart valve 200 can comprise a frame 205, a plurality of leaflets 225, and an outer skirt 230. However, other examples of the prosthetic heart valve 200 can comprise additional and/or alternative components.

The frame 205 can comprise an annular structure such as a cylinder. The circumference of the cylinder can form a central flow orifice through which blood can flow. The cylinder can be aligned such that the central flow orifice extends from an inflow end portion 215 at an inflow end of the frame 205 to an outflow end portion 210 at the outflow end of the frame 205. The frame 205 can be made of any suitable metal or other materials. In some examples, the frame 205 can comprise a metal wire or mesh frame. The metal wire or mesh frame can be configured to be radially expandable or compressible.

The frame 205 can include a plurality of commissure posts 220 (which can also be referred to as “commissure supports”) which are configured to secure the plurality of leaflets 225 to the frame 205. The plurality of commissure posts 220 can comprise a plurality of upstanding posts circumferentially distributed around the frame 205 and extending towards the outflow end portion 210. In some examples, the plurality of commissure posts 220 can be extensions of the frame 205. The plurality of commissure posts 220 can comprise any suitable metal, polymer, plastic, bioprosthetic tissue, suturing material, or other material.

The plurality of leaflets 225 can be configured to regulate blood flow through the prosthetic heart valve 200. In the depicted example, the plurality of leaflets 225 can include three leaflets arranged to collapse in a tricuspid arrangement. However, other examples of the prosthetic heart valve 200 can include the three leaflets arranged to collapse in a different arrangement or can comprise a different number of leaflets (e.g., two leaflets, etc.).

Each of the plurality of leaflets 225 can include two lateral portions 224. Each lateral portion 224 can define an edge that extends from the inflow end portion 215 towards the outflow end portion 210. The lateral portions 224 can be aligned with and secured to the plurality of commissure posts 220. For example, each of the two lateral portions 224 can be secured to an adjacent, corresponding one of the plurality of commissure posts 220.

Each of the plurality of leaflets 225 can include an inflow edge portion 226 (which can also be referred to as a cusp edge portion) extending between the two lateral portions 224 on a portion of the leaflet 225 closest to the inflow end portion 215. The inflow edge portions 226 can define an undulating, curved scallop edge that generally follows or tracks portions of the frame 205 in a circumferential direction. The inflow edge portions 226 of the plurality of leaflets 225 can also be referred to as a “scallop line.”

Each of the plurality of leaflets 225 can further include a free edge portion 228 on a portion of the leaflet 225 between the two lateral portions 224 and closest to the outflow end portion 210. The free edge portion 228 of each of the plurality of leaflets 225 can extend inwardly towards the central flow orifice and co-apt, or mate, with the free edge portions 228 of the other leaflets when the plurality of leaflets 225 are in a closed configuration, so as to occlude blood flow through the central flow orifice. When the plurality of leaflets 225 are in an open configuration, the free edge portion 228 of each of the plurality of leaflets 225 can generally follow portions of the frame 205 in the circumferential direction, so as not to occlude blood flow through the central flow orifice.

The plurality of leaflets 225 can be biased or shape-set. In some examples, the plurality of leaflets 225 can be shape-set to the open configuration. Shape-setting the plurality of leaflets 225 in the open configuration can beneficially reduce the amount of force needed to actuate the plurality of leaflets 225 from the closed configuration to the open configuration, thereby improving the actuation of the plurality of leaflets 225 between the open and closed configurations during normal valve operation. In other examples, the plurality of leaflets 225 can be shape-set to the closed configuration or an intermediate configuration between the open configuration and the closed configuration.

In some examples, the plurality of leaflets 225 can be made from a bioprosthetic tissue, such as bovine pericardium. For example, the plurality of leaflets 225 can be formed by cutting the leaflets from a pericardial sac of a cow. However, other examples of the plurality of leaflets 225 can comprise any other suitable tissue or material. The bioprosthetic tissue can be treated or dehydrated such that the prosthetic heart valve 200 can be stored in a non-aqueous environment, i.e., not stored in a liquid preservative, prior to surgical implantation. Furthermore, the bioprosthetic tissue can be sterilized to prevent microbial or fungal growth.

In some examples, the prosthetic heart valve 200 can include one or more skirts mounted around the frame 205. For example, as shown in FIG. 6, the prosthetic heart valve 200 can include the outer skirt 230 mounted around an outer surface of the frame 205. In the depicted example, the outer skirt 230 can extend from the inflow end portion 215 to the outflow end portion 210 and/or cover substantially the entire outer surface of the frame 205. In other examples, the outer skirt 230 can cover only a portion (e.g., the inflow end portion 215) of the frame 205. The outer skirt 230 can be configured to function as a sealing member for the prosthetic heart valve 200 by sealing against the tissue of the native valve annulus (or against a docking device, e.g., 52, if the prosthetic valve 200 is secured within the docking device) and helping to reduce paravalvular leakage past the prosthetic heart valve 200.

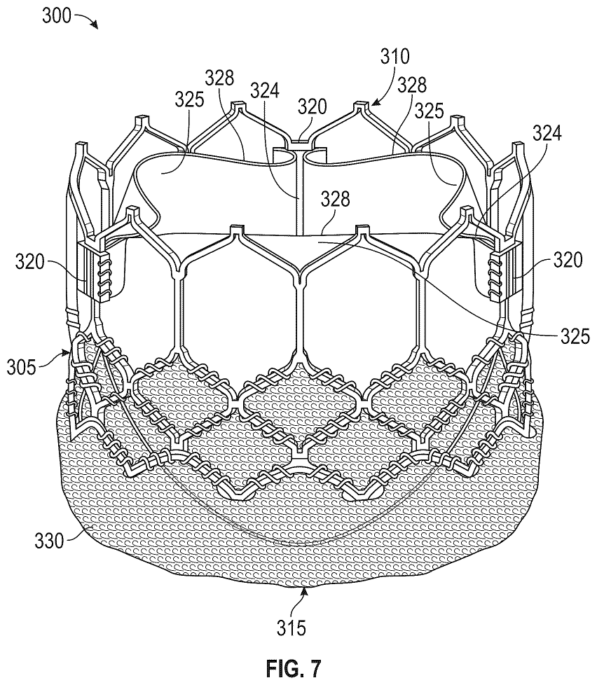

In some cases, an inner skirt (e.g., similar to 330 depicted in FIG. 7) can be mounted around an inner surface of the frame 205. The inner skirt can be configured to function as a sealing member to prevent or decrease perivalvular leakage, to anchor the plurality of leaflets 225 to the frame 205, and/or to protect the plurality of leaflets 225 against damage caused by contact with the frame 205 during crimping and during working cycles of the prosthetic heart valve 200. In some examples, the inflow edge portions of the plurality of leaflets 225 can be sutured to the inner skirt generally along the scallop line. The inner skirt can in turn be sutured to the frame 205. In other examples, the plurality of leaflets 225 can be sutured directly to the frame 205.

The inner and outer skirts can be formed from any of various suitable biocompatible materials, including any of various synthetic materials, including fabrics (e.g., polyethylene terephthalate fabric) or natural tissue (e.g., pericardial tissue).

In certain examples, an identification tag (not shown) can be secured to the frame 205, e.g., with a suture. The identification tag can provide a serial number representative of information regarding the type of heart valve and other particularities about its manufacture, such as the date.

FIG. 7 depicts an example prosthetic heart valve 300, according to another example. As shown, the prosthetic heart valve 300 can comprise a frame 305, a plurality of leaflets 325, and an inner skirt 330. The frame 305 can include a plurality of commissure posts 320 configured to secure the plurality of leaflets 325. Although three leaflets 325 are shown in FIG. 7, it is to be understood that the prosthetic heart valve 300 can have different number of leaflets (e.g., two leaflets). Other examples of the prosthetic heart valve 300 can comprise additional or alternative components.

In the illustrated example, each of the plurality of leaflets 325 can comprise a free edge portion 328 disposed towards an outflow end portion 310 of the prosthetic heart valve 300, wherein the free edge portion is between two lateral portions 324. Each of the two lateral portions 324, which extend between an inflow end portion 315 and an outflow end portion 310 of the frame 305, can be secured to corresponding adjacent ones of the plurality of commissure posts 320 while the free edge portion 328 can remain unattached to the plurality of commissure posts 320 or the frame 305.

The inner skirt 330 can be mounted around an inner surface of the frame 305. Similarly, the inner skirt 330 can be configured to function as a sealing member to prevent or decrease perivalvular leakage, to anchor the plurality of leaflets 325 to the frame 305, and/or to protect the plurality of leaflets 325 against damage caused by contact with the frame 305 during crimping and during working cycles of the prosthetic heart valve 300. Although not shown, the prosthetic valve 300 can include an outer skirt (e.g., similar to 230 depicted in FIG. 6) mounted around an outer surface of the frame 305.

In the illustrated example, each of the plurality of leaflets 325 can be biased or shape-set to the open configuration, in which the free edge portion of each of the plurality of leaflets 325 can generally follow or track the frame 305 in a circumferential direction. In some examples, each of the plurality of leaflets 325 can form a generally bell-shaped curve, wherein the tip of the bell-shaped curve can extend outwardly toward the circumference of the frame 305 and each end portion of the bell-shaped curve can terminate at one of the plurality of commissure posts 320.

In some examples, in which the prosthetic heart valve 300 is implanted at a native mitral annulus, biasing or shape-setting the plurality of leaflets 325 in the open configuration can beneficially reduce the amount of force needed to open the plurality of leaflets 325, thereby facilitating the actuation of the plurality of leaflets 325 from the closed configuration to the open configuration.

However, in other examples, the prosthetic heart valve 300 can be implanted in an aortic annulus or any other suitable annulus in a patient's heart. If the prosthetic heart valve 300 is implanted in the aortic annulus, the plurality of leaflets 325 can be biased or shape-set to a closed configuration, in which the free edge portions of each of the plurality of leaflets 325 extend into a central flow orifice formed by the frame 305. The plurality of leaflets 325 in the closed configuration can occlude blood flow through the central flow orifice formed by the frame 305. In these examples, biasing or shape-setting the plurality of leaflets 325 in the closed configuration can help counteract the pressure gradient across the aortic annulus that tends to force the plurality of leaflets 325 to the open configuration.

In any of the examples described herein, the prosthetic heart valve 200 and/or 300 can be directly implanted at a target implantation site. In other examples, the prosthetic heart valve 200 and/or 300 can be deployed and secured within a docking device (e.g., 52) which is implanted at the target implantation site. For example, the prosthetic heart valve 200 and/or 300 can be implanted (with or without a docking device) at the native mitral annulus, the native aortic annulus, the tricuspid annulus, the pulmonary annulus, and/or other native valve sites.

Additional details of various prosthetic heart valves and their components are described, for example, in U.S. Pat. Nos. 9,393,110, 9,339,384, 9,155,619, 8,652,202, and 6,730,118, U.S. Patent Publication Nos. 2020/0352711, 2019/0374337, 2019/0192296, 2019/0046314, 2018/0206982, 2018/0153689, and PCT Patent Application Publication Nos. WO/2021/188476 and WO/2020/247907, the disclosures of which are incorporated herein in their entireties for all purposes.

### Exemplary Delivery Apparatus

FIGS. 8-12 illustrate an example delivery apparatus 400 configured to deliver an implant device (e.g., a prosthesis such as the docking devices 52 and 100 described above) to a target implantation site (e.g., a heart and/or native valve of an animal, human, cadaver, and/or the like). In some examples, the delivery apparatus 400 can be a transcatheter delivery apparatus that can be used to guide the delivery of a docking device 401 through a patient's vasculature, as explained above with reference to FIGS. 1-4. The docking device 401 can be the docking device 100 described above with reference to FIG. 5. In other examples, the delivery apparatus 400 can be used to guide the delivery of a prosthetic valve (e.g., 200, 300) or other prosthetic implants.

Referring again to FIGS. 8-12, the delivery apparatus 400 can include a pusher shaft 402, a sleeve shaft 404, and a delivery shaft 406, which are all coaxial about a central longitudinal axis 408.

As shown in FIG. 8, the pusher shaft 402 can extend through an inner lumen 406i of the delivery shaft 406. The delivery shaft 406 can surround at least a distal portion of the pusher shaft 402. When navigating through the patient's vasculature, the docking device 401 can be retained at a distal end portion of the delivery shaft 406 in a substantially straight delivery configuration. The pusher shaft 402 can be positioned proximal to docking device 401. A distal end 402d of the pusher shaft 402 can abut or be spaced away from the docking device 401. After reaching the target implantation side, the pusher shaft 402 can be used to deploy the docking device 401 from the delivery shaft 406. Without the constraint of the delivery shaft 406, the docking device 401 can return to the deployed configuration, as described above.

In certain examples, deploying the docking device 401 from the delivery shaft 406 can be accomplished by manipulating the pusher shaft 402 in the distal direction while holding the delivery shaft 406 in place, by retracting the delivery shaft 406 in the proximal direction while holding the pusher shaft 402 in place, or by pushing the pusher shaft 402 in the distal direction while simultaneously retracting the delivery shaft 406 in the proximal direction. As a result, the docking device 401 can be pushed out of a distal end of the delivery shaft 406, thus changing from the delivery configuration to the deployed configuration.

In certain examples, the delivery apparatus 400 can further include a dock sleeve 418 connected to or being a distal part of the sleeve shaft 404. The dock sleeve 418 can have a lubricous outer surface. The dock sleeve 418 can be configured to cover the docking device 401 while retained inside the delivery shaft 406. In some examples, the dock sleeve 418 can have a tubular structure which has an inner diameter sufficient to surround the docking device 401 and an outer diameter that is small enough to be retained within and axially movable within the delivery shaft 406. The length of the dock sleeve 418 can be configured to cover at least the full length of the docking device 401.

In certain examples, when deploying the docking device 401 from the delivery shaft 406, the pusher shaft 402 and the sleeve shaft 404 can be configured to move together (at least initially), in the axial direction, with the docking device 401. For example, actuation of the pusher shaft 402, to push against the docking device 401 and move it out of the delivery shaft 406 can also cause the sleeve shaft 404 to move along with the pusher shaft 402 and the docking device 401. As such, the docking device 401 can remain being covered by the dock sleeve 418 during the procedure of pushing the docking device 401 into position at the target implantation site via the pusher shaft 402. Thus, when the docking device 401 is initially deployed at the target implantation site, the lubricous dock sleeve 418 can facilitate the covered docking device 401 to encircle the native anatomy.

The pusher shaft 402 and the sleeve shaft 404 can also be actuated independently of each other. For example, the sleeve shaft 404 can be axially extended and/or collapsed over the pusher shaft 402, as described further below. Thus, after confirming the docking device 401 is desirably positioned at the target implantation site, the dock sleeve 418 can be retracted proximally so as to expose the docking device 401.

As described further below, the dock sleeve 418 can be connected to the sleeve shaft 404 so that axially extending or retracting the sleeve shaft 404 can cause the dock sleeve 418 to move distally to cover the docking device 401 or move proximally to uncover the docking device 401. In some examples, the sleeve shaft 404 and the dock sleeve 418 can be fixed in position. In such circumstances, the docking device 401 can be moved distally or proximally relative to the sleeve shaft 404 so that the docking device is uncovered or covered by the dock sleeve 418, respectively. In certain examples, the dock sleeve 418 can be an integral part (e.g., a distal end portion) of the sleeve shaft 404.

As described herein, the sleeve shaft 404 can comprise a telescopic section which includes a plurality of nested shaft members 412, 414 (which can also be referred to as “shaft segments”). Specifically, the sleeve shaft 404 can include a fixed shaft member 412 that is fixedly mounted (e.g., by means of welding, adhesive, mechanical fasteners, or the like) around a proximal portion 402p of the pusher shaft 402. The sleeve shaft 404 can also include one or more movable shaft members 414a, 414b, 414c, 414d, etc. (collectively 414) that are coaxial with and axially movable relative to the fixed shaft member 412 and the pusher shaft 402.

As shown in FIGS. 8-12, the delivery shaft 406 can be positioned distal to the fixed shaft member 412 of the sleeve shaft 404. In certain examples, a proximal end 412p of the fixed shaft member 412 can be positioned distal to a proximal end 402p of the pusher shaft 402. In certain examples, at least some of the one or more movable shaft members 414 are configured to be axially extendable into the inner lumen 406i of the delivery shaft 406 (see, e.g., FIGS. 8, 9A, 10A, and 12A-12B).

In the example depicted in FIG. 8-12, the fixed shaft member 412 is an outer-most shaft member (which can also be referred to as an “outer shaft member”), and the one or more moveable shaft members 414 are inner shaft members that can be retained within the fixed shaft member 412, i.e., the fixed shaft member 412 has a larger inner diameter than the one or more movable shaft members 414 (at least along a majority of its length).

As shown, the sleeve shaft 404 can include an inner-most shaft member 414a which has the smallest diameter of the movable shaft members. The pusher shaft 402 can extend through an inner lumen 415 of the inner-most shaft member 414a. In the depicted example, the sleeve shaft 404 includes one or more intermediate shaft members (e.g., 414b, 414c, 414d, etc.) that are coaxially arranged between the inner-most shaft member 414a and the fixed shaft member 412. In other examples, the sleeve shaft 404 can include only one fixed shaft member (e.g., 412) and one movable shaft member (e.g., 414a) without an intermediate shaft member arranged therebetween.

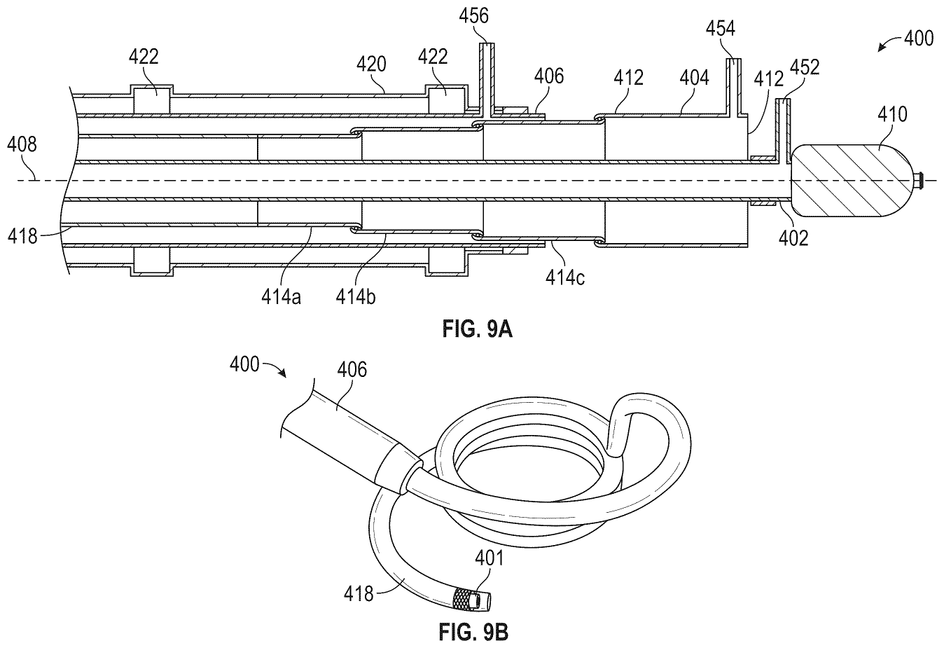

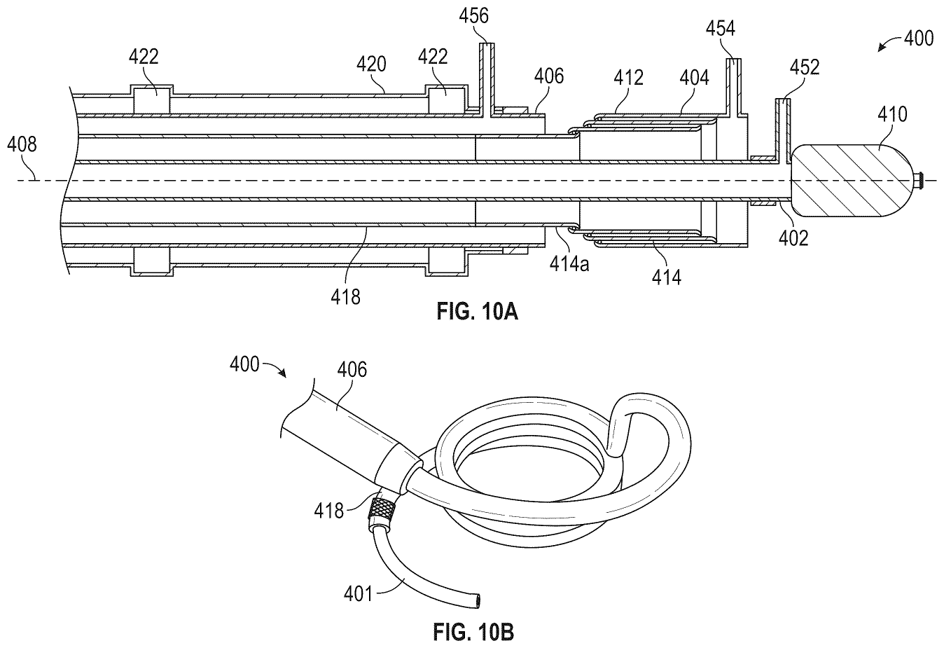

The delivery apparatus 400 can include an actuation mechanism (which is described more fully below) configured to axially move the one or more movable shaft members 414 relative to the fixed shaft member 412 so that the sleeve shaft 404 can axially move between an extended configuration and a collapsed configuration. As examples, FIGS. 9A-9B show the sleeve shaft 404 in the fully extended configuration, FIGS. 11A-11C show the sleeve shaft 404 in the collapsed configuration, and FIGS. 10A-10B shows the sleeve shaft 404 in an intermediate state, or partially extended configuration. Thus, the axial length of the sleeve shaft 404 can progressively increase from the collapsed configuration to the partially extended configuration and then to the fully extended configuration.

As shown in FIG. 11A, when the sleeve shaft 404 is in the collapsed configuration, each of the one or more movable shaft members 414 can be substantially received within the fixed shaft member 412. As described herein, a movable shaft member is deemed to be substantially received within the fixed shaft member 412 when an axial length of the movable shaft member being covered by the fixed shaft member 412 is greater than a predefined percentage (e.g., 80%, 90%, 95%, etc.) of an axial length of the movable shaft member. As an example, when the sleeve shaft 404 is in the collapsed configuration, each of the movable shaft members 414 can be completely retained within the fixed shaft member 412 so that no movable shaft member is exposed. As another example, when the sleeve shaft 404 is in the collapsed configuration, only the distal tips/ends of respective movable shaft members 414 may be exposed while the main body portions of respective movable shaft members 414 are retained within the fixed shaft member 412.

As shown in FIG. 9A, when the sleeve shaft 404 is in the fully extended configuration, each of the one or more movable shaft members 414 can substantially extend out of the fixed shaft member 412, e.g., the one or more movable shaft members 414 can be configured such that a movable shaft member having a smaller diameter extends to a more distal position than another movable shaft member having a larger diameter.

In certain examples, each of the one or more movable shaft members 414 can have a lubricant coating over its outer surface. In certain examples, the fixed shaft member 412 and the intermediate shaft members (e.g., 414b, 414c, 414d, etc.) can have lubricant coatings on respective inner surfaces. The lubricant coatings can facilitate axial movement the movable shaft members 414.

In certain examples, the dock sleeve 418 can be fixedly connected or bonded to a distal end 413 of the inner-most shaft member 414a. In certain examples, the dock sleeve 418 can overlaps with a portion of the inner-most shaft member 414a (e.g., a proximal end portion of the dock sleeve 418 can cover the distal end 413 the inner-most shaft member 414a). In some examples, the dock sleeve 418 can have about the same diameter as the inner-most shaft member 414a. In other examples, the dock sleeve 418 can have a larger or smaller diameter than the inner-most shaft member 414a. In certain examples, the dock sleeve 418 can be an integral part (e.g., a distal end portion) of the inner-most shaft member 414a.

Thus, by axially extending or shortening the sleeve shaft 404 (e.g., by axially moving the one or more movable shaft members 414), the dock sleeve 418 can cover or uncover the docking device 401. Alternatively, when the sleeve shaft 404 and the dock sleeve 418 are fixed in position, advancing the docking device 401 in the distal direction or retracting the docking device 401 in the proximal direction can cause the docking device 401 to be uncovered or covered by the dock sleeve 418, respectively.

For example, FIG. 9B shows the docking device 401 initially deployed out of the delivery shaft 406. In this configuration, the sleeve shaft 404 is in the fully extended configuration as depicted in FIG. 9A, and the docking device 401 is covered by the dock sleeve 418. FIG. 10B shows the deployed docking device 401 that is partially covered by the dock sleeve 404. In this configuration, the sleeve shaft 404 is in the partially extended configuration as depicted in FIG. 10A. As a result, a distal portion of the docking device 401 is exposed whereas a proximal portion of the docking device 401 remains being covered by the dock sleeve 418. FIG. 11B shows the deployed docking device 401 that is completely uncovered by the dock sleeve 418. In this configuration, the sleeve shaft 404 is in the collapsed configuration as depicted in FIG. 11A. As a result, the dock sleeve 418 is retracted back into the delivery shaft 406, thereby exposing the docking device 401, as well as a release suture 403 connected to the docking device 401.

In certain examples, the dock sleeve 418 can be configured to be flexible and/or have a lower durometer than the movable shaft members 414. If the dock sleeve 418 is an integral or a distal end portion of the inner-most shaft member 414a, the dock sleeve 418 can be configured to have a lower durometer than a proximal end portion of the inner-most shaft member 414a. In certain examples, the shaft members 412, 414 can comprise a relatively more rigid metal material (e.g., stainless steel, etc.) and constructed as hypotubes. The dock sleeve 418 can comprise a polymeric material such as low durometer thermoplastic elastomers (e.g., PTFE, chronoprene, santoprene, tecothane, etc.). In certain examples, the dock sleeve 418 can be bonded to the inner-most shaft member 414a through a reflow process. In certain examples, the dock sleeve 418 can have a hydrophilic coating, which can act as a lubricous surface to improve the ease of encircling the native anatomy and reduce risk of damage to the native tissue.

Additional examples of the dock sleeve 418 are described further in Provisional U.S. Application No. 63/138,910, the entirety of which is incorporated by reference herein.

Alternatively, the fixed shaft member can have a smaller diameter than the one or more movable shaft members. For example, the inner-most shaft member can be the fixed shaft member while the outer-most shaft member and intermediate shaft members (if any) can be movable shaft members. In such cases, the pusher shaft 402 can extend through a lumen of the fixed shaft member. The dock sleeve 418 can be connected to a distal end of the outer-most shaft member. In certain examples, the dock sleeve 418 can be an integral part (e.g., a distal end portion) of the outer-most shaft member. Similarly, the one or movable shaft members surrounding the fixed shaft member can be progressively moved to more distal positions relative to the fixed shaft member (i.e., move to the extended configuration) or be moved proximally so that the movable shaft members substantially surround the fixed shaft member (i.e., move to the collapsed configuration). Also, although the examples in FIGS. 8-12 show the outer-most shaft member as the fixed shaft member and inner shaft members as movable shaft members, it should be understood that the same principles described herein also apply when the inner-most shaft member is the fixed shaft member and the outer shaft members are movable shaft members.

In certain examples, the delivery apparatus 400 can include a suture lock 410 connected to the proximal end 402p of the pusher shaft 402. The suture lock 410 can be configured to be releasably connected to the docking device 401, e.g., via the release suture 403 extending through a lumen 402i of the pusher shaft 402. The suture lock 410 can also include a cutting mechanism configured to cut the release suture 403 to release, detach, decouple, and/or otherwise disconnect the docking device 401 from the delivery apparatus 400.

As shown in FIGS. 8-12, the suture lock 410 can be configured to be in-line with the pusher shaft 402 and the sleeve shaft 404. In certain examples, the suture lock 410 can be releasably connected to the pusher shaft 402 via a lock and release mechanism, such as a rotatable release knob. When connected, the axial distance between the suture lock 410 and the fixed shaft member 412 can be fixed.

Additional details on a suture lock are described in International Patent Applications No. PCT/US2020/36577 and PCT/US2021/056150. Additional examples of the lock and release mechanism for a suture lock are described in Provisional U.S. Patent Appl. Nos. 63/362,996 and 63/366,733, which are incorporated by reference herein in their entireties.

As shown in FIGS. 9A, 10A, 11A, and 12A-12B, the delivery apparatus 400 can further include a handle 420 (omitted in FIG. 8 for simplicity). The pusher shaft 402 can extend through the handle 420. A proximal portion of the delivery shaft 406 can be connected to the handle 420. In certain examples, a proximal end 406p of the delivery shaft 406 can extend proximally out of the handle 420. In other examples, the proximal end 406p of the delivery shaft 406 can terminate inside the handle 420.

Because the fixed shaft member 412 is fixedly mounted on the pusher shaft 402, as long as the pusher shaft 402 is not axially moved relative to the handle 420, the axial distance between the suture lock 410 and the handle 420 can also remain fixed (or at least substantially fixed). Thus, the telescopic movement of the movable shaft members 414 relative to the fixed shaft member 412 does not affect the overall length of the delivery apparatus 400.

The fixed shaft member 412 can be positioned proximal to the handle 420, and the one or more movable shaft members 414 can be configured to be axially extendable into the handle 420. For example, when the sleeve shaft 404 is in the axially extended configuration, the inner-most shaft member 414a can extend into the handle 420 (and also into the inner lumen 406i of the delivery shaft 406). When the sleeve shaft 404 is in the axially collapsed configuration, the inner-most shaft member 414a can be positioned proximal to the handle 420.

In certain examples, the handle 420 can include one or more actuators configured to adjust position and/or curvature of the pusher shaft 402, the sleeve shaft 404, and/or the delivery shaft 406. For example, the handle 420 can include one or more rotatable knobs 422 (or buttons, wheels, or the like) configured to selectively flex a distal portion of the delivery shaft 406 so as to facilitate navigating the delivery shaft 406 through the patient's vasculature during the implantation process.

The handle 420 can further include one or more actuation mechanism configured to adjust axial positions of the pusher shaft 402, the sleeve shaft 404, and/or the delivery shaft 406 relative to one another. For example, as described more fully below, the handle 420 can include an actuation mechanism configured to move the sleeve shaft 404 between the axially collapsed configuration and the axially extended configuration.

As shown in FIGS. 8-12, various components of the delivery apparatus 400, including the pusher shaft 402, the sleeve shaft 404, the delivery shaft 406, the handle 420, and the suture lock 410 can all be arranged to extend along the longitudinal axis 408.

In certain examples, the delivery apparatus 400 can include one or more flushing ports (e.g., 452, 454, 456) configured to supply flushing and/or anticoagulant fluid to different portions of the delivery apparatus 400. In certain examples, at least some of the flushing ports (e.g., 456) can be connected and/or located on the handle 420, as described further below.

Additional details on the handle of a delivery apparatus are described in International Application Nos. PCT/US2020/36577 and PCT/US2021/056150.

### Exemplary Actuation Mechanisms

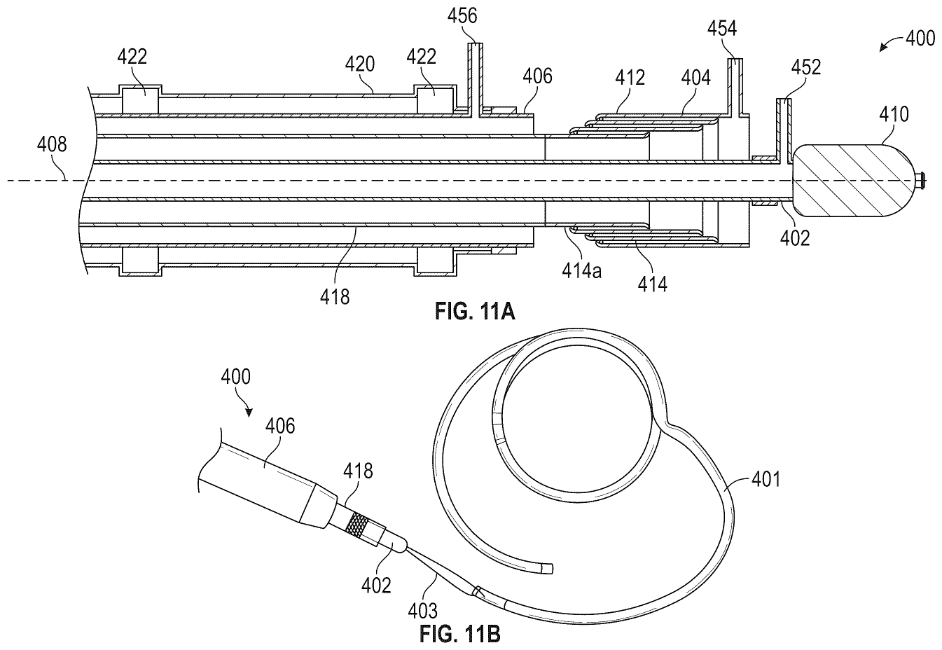

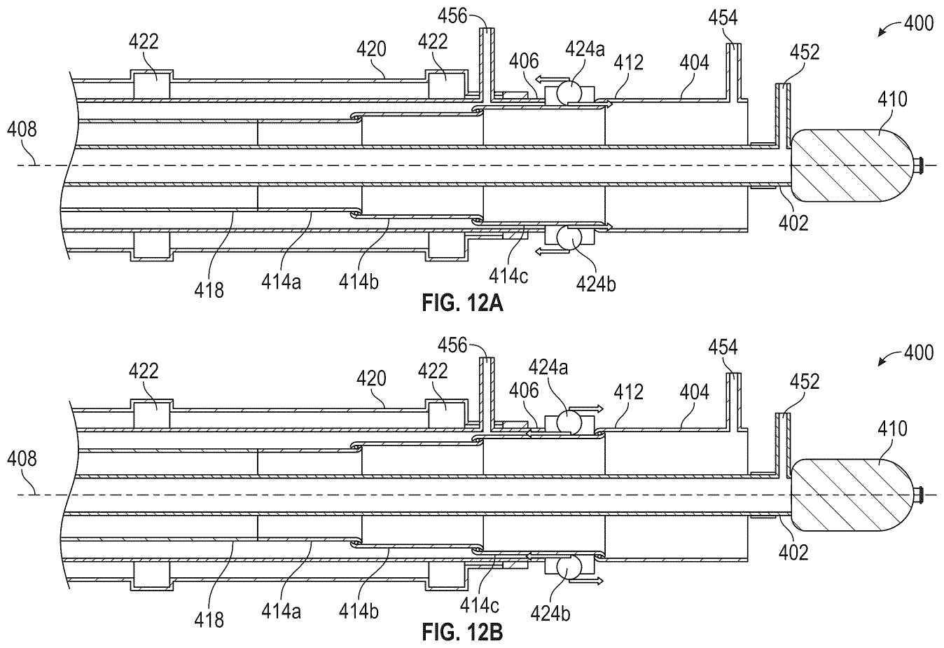

FIGS. 12-14 describe several exemplary actuation mechanisms that can successively or telescopically move the one or more movable shaft members 414 in a distal direction (e.g., toward the distal end 402d of the pusher shaft 402) relative to the fixed shaft member 412 so that the dock sleeve 418 can cover the docking device 401. In addition, the depicted actuation mechanisms can successively or telescopically move/retract the one or more movable shaft members 414 in a proximal direction relative to the fixed shaft member 412 so that the dock sleeve 418 can be removed from the docking device 401. The actuation mechanisms can be mounted on the handle 420. In certain examples, any of the actuation mechanisms described herein can include a motor operatively coupled to the one or more movable shaft members 414. In other examples, the actuation mechanisms can be manually operated without a motor.

For example, by operating the actuation mechanism, the one or more movable shaft members 414 can be successively moved in a distal direction so that distal ends of the one or more movable shaft members 414 are moved to progressively more distal positions along and over the pusher shaft 402. As a result, the dock sleeve 418 can also be moved distally to cover the docking device 401 (see, e.g., FIGS. 9A-9B). Conversely, the actuation mechanism can be operated to move the one or more movable shaft members 414 in a proximal direction until they are substantially received within the fixed shaft member 412. As a result, the dock sleeve 418 can also be moved proximally to uncover the docking device 401 (see, e.g., FIGS. 11A-11B).

In certain examples, the actuation mechanism can include at least one rotational actuator operatively connected to the one or more movable shaft members 414. Rotating the at least one rotational actuator in a first direction can telescopically move the sleeve shaft 404 from the axially collapsed configuration to the axially extended configuration, whereas rotating the at least one rotational actuator in a second direction opposite to the first direction can telescopically move the sleeve shaft from the axially extended configuration to the axially collapsed configuration.

In certain examples, the at least one rotational actuator can be configured to rotationally engage the sleeve shaft 404, e.g., the one or more movable shaft members 414, at a location distal to the fixed shaft member 412.

As one example, FIGS. 12A-12B show two rollers 424a, 424b that function as rotational actuators. Although two rollers 424a, 424b are shown in this example, the delivery apparatus 400 can include more or less than two rollers in other instances. In certain examples, the rollers 424a, 424b can be positioned distal to the fixed shaft member 412 and configured to frictionally engage respective outer surfaces of the movable shaft members 414.

As depicted in FIG. 12A, positioning the rollers 424a, 424b in contact with a movable shaft member (e.g., the movable shaft members 414 and rotating the roller 424a in a counterclockwise direction and rotating the roller 424b in a clockwise direction (as indicated by the arrows) can move the movable shaft member proximally relative to the fixed shaft member 412, thus moving the sleeve shaft 404 proximally relative to the pusher shaft 402. Conversely, as depicted in FIG. 12B, positioning the rollers 424a, 424b in contact with a movable shaft member and rotating the roller 424b in a clockwise direction and/or rotating the roller 424b in a counterclockwise direction (as indicated by the arrows) can move the movable shaft member distally relative to the fixed shaft member 412, thus moving the sleeve shaft 404 distally relative to the pusher shaft 402.

The one or more movable shaft members 414 can be moved sequentially. For example, to move the sleeve shaft 404 from the collapsed configuration to the extended configuration, the rollers 424a, 424b can first frictionally engage and move the inner-most shaft member 414a in the distal direction. After the inner-most shaft member 414a is fully extended, the rollers 424a, 424b can frictionally engage and move an intermediate shaft member (e.g., 414b, 414c, 414d, etc.) immediately surrounding the inner-most shaft member 414a in the distal direction. The rollers 424a, 424b can then frictionally engage and move other nested intermediate shaft members (if any) in the distal direction, one by one (e.g., following the sequence of increasing diameter of the shafter members), until all movable shaft members 414 respectively extend to their most distal positions.

Conversely, to move the sleeve shaft 404 from the extended configuration to the collapsed configuration, the rollers 424a, 424b can first frictionally engage and move an intermediate shaft member immediately surrounded by the fixed shaft member 412 in the proximal direction. After that intermediate shaft member is retracted into the fixed shaft member 412, the rollers 424a, 424b can then frictionally engage and move other nested intermediate shaft members (if any) and the inner-most shaft member 414a, one by one (e.g., following the sequence of decreasing diameter of the shafter members), until all movable shaft members 414 are substantially received within the fixed shaft member 412.

The rollers 424a, 424b can be configured to accommodate movable shaft members 414 of various diameters. In certain examples, a biasing mechanism can be coupled to the rollers 424a, 424b so that when the rollers 424a, 424b are in the unbiased state, the radial distance between the rollers 424a, 424b is about the same or smaller than the outer diameter of the inner-most shaft member 414a. The biasing mechanism can urge the rollers 424a, 424b radially inward toward the outer diameter of any movable shaft member 414 located thereof.

For example, when moving the inner-most shaft member 414a, the rollers 424a, 424b can engage the outer surface of the inner-most shaft member 414a under the biasing force. When moving an intermediate shaft member (e.g., 414b, 414c, etc.), which has a larger outer diameter than the innermost shaft member 414a, the rollers 424a, 424b can be urged to a biased state due to an increased radial distance between the rollers 424a, 424b. As a result, the rollers 424a, 424b can press radially inwardly against the outer surface of the intermediate shaft member under the biasing force. Thus, the rollers 424a, 424b can remain contact with the outer surfaces of the movable shaft members 414 regardless of their various diameters.

In certain examples, the biasing mechanism can include springs respectively directly attached to the rollers 424a, 424b. In other examples, the biasing mechanism can include springs attached to respective arms, which are further connected to the rollers 424a, 424b.

The rotational actuator can take other forms so long as it can transform rotational movement of one object into axial movement of the one or more movable shaft 414.

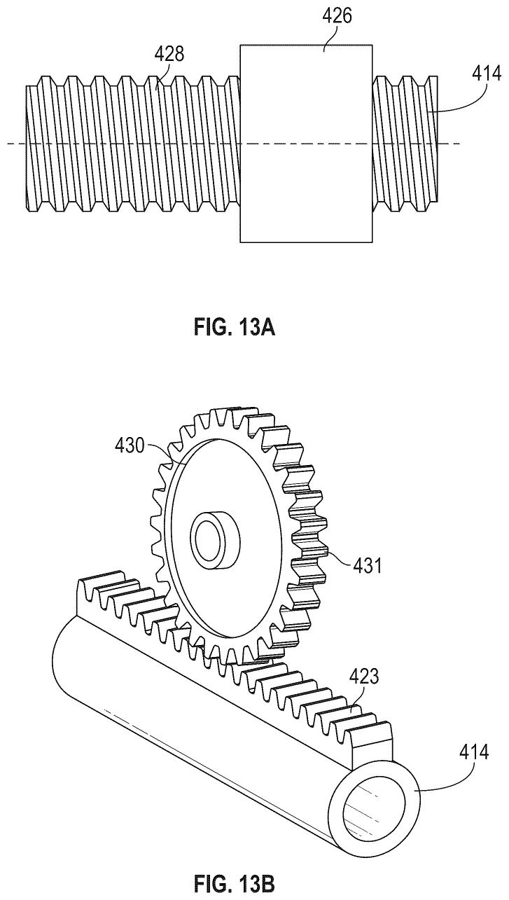

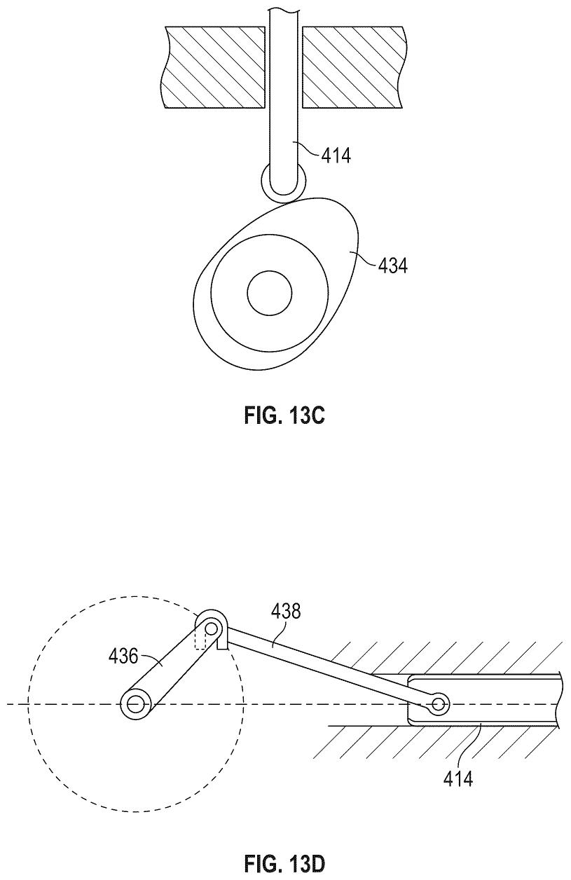

As an example, FIG. 13A shows a nut 426 which can be configured as a rotational actuator. Each of the movable shaft members 414 can have external threads 428 configured to matingly engage with internal threads of the nut 426. The nut 426 can be configured to be free to rotate but axially fixed in a position distal to the fixed shaft member 412. Thus, the moveable shaft members 414 can function as lead screws, and rotation of the nut 426 can cause corresponding axial movement of the movable shaft members 414. In some examples, the nut 426 can have a resiliently constricted inner bore so that the nut 426 can threadably engage the one or more movable shaft members 414 having different diameters. For example, the inner bore of the nut 426 can be enlarged to threadably engage a movable shaft member having a larger diameter, or vice versa.

As another example, FIG. 13B shows a circular gear 430 (also referred to as a “pinion gear”) which can be configured as a rotational actuator. Each of the movable shaft members 414 can have external teeth 432 configured to matingly engage with teeth 431 of the gear 430. The gear 430 can be configured to be free to rotate but axially fixed in a position distal to the fixed shaft member 412. Thus, the moveable shaft members 414 can function as linear gears or racks, and rotation of the gear 430 can cause corresponding axial movement of the movable shaft members 414. In some examples, the gear 430 can be configured to bias radially inwardly toward to pusher shaft 402 so that gear 430 can matingly engage the one or more movable shaft members 414 having different diameters. For example, the gear 430 can move slightly closer to the pusher shaft 402 to matingly engage a movable shaft member having a smaller diameter, or vice versa.

As another example, FIG. 13C shows a cam 434 that can be configured as a rotational actuator. The cam 434 can be an eccentric disk or have other shapes and configured to contact a proximal end of a movable shaft member (e.g., 414). Thus, rotatory motion of the cam 434 can be transformed into axial motion of the movable shaft member.

As yet another example, FIG. 13D shows a rotatable drive shaft 436 that can be configured as a rotational actuator. A rod 438 can have a first end hingedly connected to the drive shaft 436 and another end connected one of the movable shaft members (e.g., 414). Thus, rotation motion of the drive shaft 436 can be converted to axial movement of the movable shaft member.

In certain examples, the actuation mechanism can include at least one linear actuator operatively connected to the one or more movable shaft members 414. Moving or translating the at least one linear actuator in a first direction can telescopically move the sleeve shaft 404 from the axially collapsed configuration to the axially extended configuration, whereas moving or translating the at least one linear actuator in a second direction opposite to the first direction can move the sleeve shaft 404 from the axially extended configuration to the axially collapsed configuration.

The linear actuator can take a variety of forms so long as linear movement of one object can cause axial movement of the one or move movable shaft members 414.

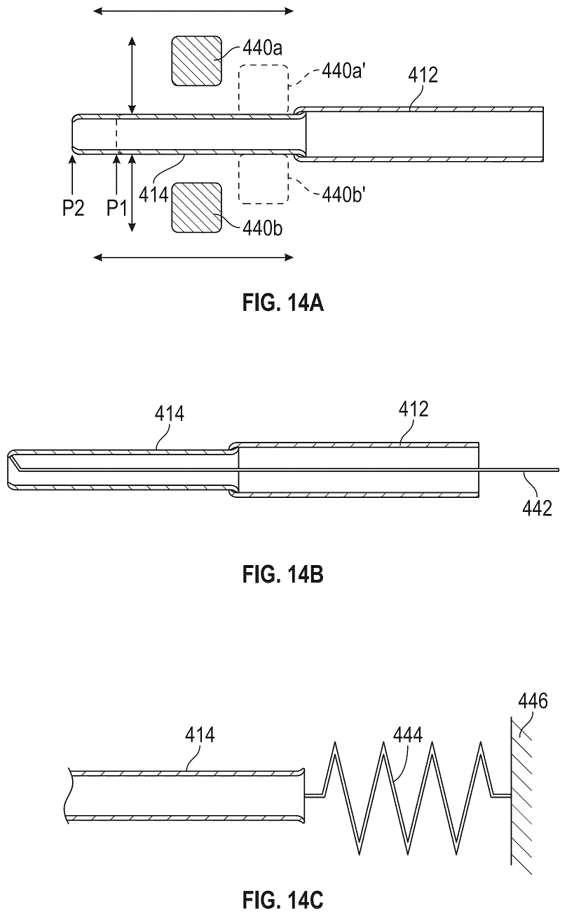

As one example, FIG. 14A shows two grippers 440a, 440b that can function as linear actuators. Although two grippers 440a, 440b are shown in this example, the delivery apparatus 400 can include more or less than two grippers in certain instances. In certain examples, the grippers 440a, 440b can be positioned distal to the fixed shaft member 412 and can be axially movable relative to the fixed shaft member 412.

The grippers 440a, 440b can be configured to frictionally engage an outer surface of a movable shaft member 414 when the grippers 440a, 440b are radially compressed (as illustrated by the dashed 440a′, 440b′ in FIG. 14A). The grippers 440a, 440b can also be detached from the movable shaft member (as illustrated by the solid 440a, 440b in FIG. 14A) when the grippers 440a, 440b are not radially compressed (e.g., through a bias spring mechanism or the like). Radial movement of the grippers 440a, 440b are indicated by the vertical arrows in FIG. 14A.

Thus, by pressing the grippers 440a, 440b radially inwardly until they contact the outer surface of a movable shaft member and then move the grippers 440a, 440b in the distal or proximal direction, the movable shafter member can also be moved in the distal or proximal direction. After releasing the pressure, the grippers 440a, 440b can be detached from the movable shaft member and moved to another axial position to move another movable shaft member. Axial movement of the grippers 440a, 440b are indicated by the horizontal arrows in FIG. 14A. For example, the distal end of the shaft 414 can move from P1 to P2 when the grippers 440a, 440b grip the shaft 414 and move in the distal direction, or reversely from P2 to P1 when the grippers 440a, 440b grip the shaft 414 and move in the proximal direction.

As another example, FIG. 14B shows a slider 442 that can be configured as a linear actuator. In certain examples, the slider 442 can be a rigid thin rod that is fixedly connected to a movable shaft member (e.g., 414) and is axially movable relative to the fixed shaft member 412. Thus, by pushing the slider 442 in the distal direction or pulling the slider 442 in the proximal direction, the movable shaft member can also be moved in the distal or proximal direction accordingly.

In certain examples, the actuation mechanism can include at least one bias member operatively connected to the one or more movable shaft members 414. In certain examples, the bias member can be configured to resiliently bias a movable shaft member (e.g., 414) axially toward the fixed shaft member 412 if the movable shaft member is positioned distally relative to the fixed shaft member. In such a case, the sleeve shaft 404 is biased to the collapsed configuration. In other examples, the bias member can be configured to resiliently bias a movable shaft member (e.g., 414) axially away from the fixed shaft member 412 if the movable shaft member is positioned/nested within the fixed shaft member 412. In such a case, the sleeve shaft 404 is biased to the extended configuration.

As one example, FIG. 14C shows a spring 444 which can function as a bias member. In certain examples, one end of the spring 444 can be connected to a proximal end of a movable shaft member (e.g., 414), and another end of the spring 444 can be connected to a component 446 within the handle 420. When the spring 444 is in the unbiased state, the spring 444 can have a resting or equilibrium length.

In certain examples, the spring 444 can be moved to the biased state by axially extending the spring 444 so that it has a longer length than the resting length. Axial extension of the spring 444 can push the connected movable shaft member in the distal direction. When the spring 444 returns to the unbiased state (i.e., returns to its resting length), the connected movable shaft member can be pulled in the proximal direction.

In other examples, the spring 444 can be moved to the biased state by axially compressing the spring 444 so that it has a shorter length than the resting length. Axial compression of the spring 444 can pull the connected movable shaft member in the proximal direction. When the spring 444 returns to the unbiased state (i.e., returns to its resting length), the connected movable shaft member can be pushed in the distal direction.

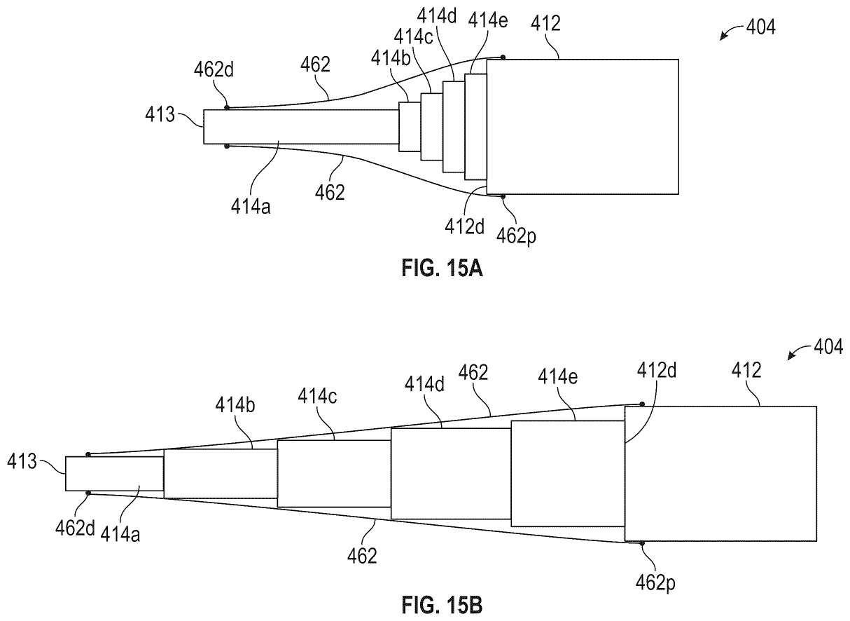

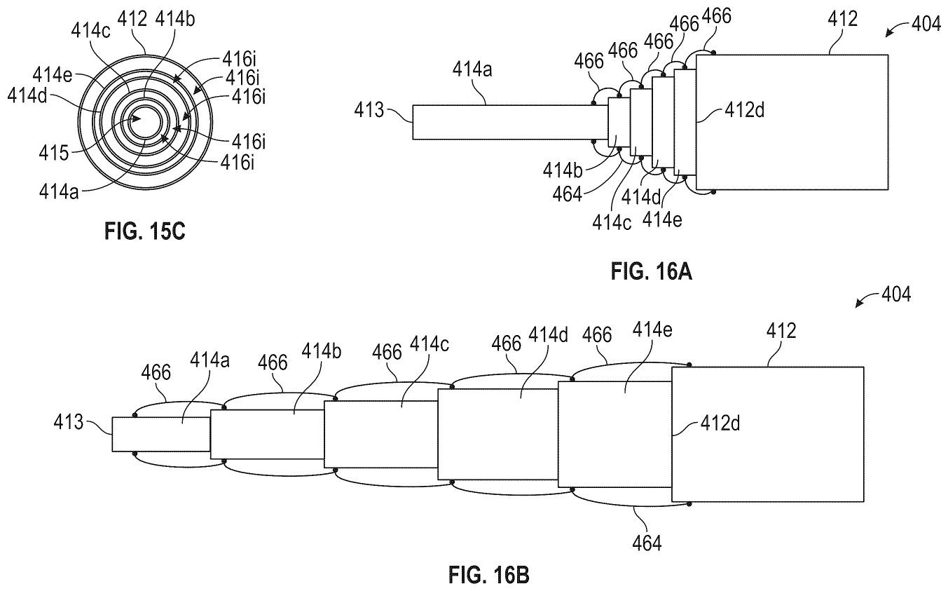

In still other examples, the bias member can be configured as, and/or coupled with, a sealing member (e.g., 462, 464), as described further below with reference to FIGS. 15A-15B and 16A-16B.

In certain examples, the sleeve shaft 404 can further include a locking mechanism configured to lock the sleeve shaft 404 in the axially extended configuration. Such locking mechanism can prevent an axially extended sleeve shaft 404 from accidently collapsing into the axially collapsed configuration, e.g., when moving the pusher shaft 402 and the sleeve shaft 404 together during initial deployment of the docking device 401.

In some examples, the locking mechanism can be based on inherent friction force existing between the movable shaft members 414. For example, the sealing members (e.g., 462, 464) described above can frictionally resist relative axial movement between the movable shaft members 414.

In some examples, the slider 442 depicted in FIG. 14B can be configured as a locking mechanism. For example, by holding the slider 442 stationary relative to the fixed shaft member 412, axial positions of the movable shaft members 414 can be fixed relative to the fixed shaft member 412.

In some examples, the locking mechanism can include a peg-and-slot configuration. For example, a first movable shaft member can have a radially protruding peg received in a slot of a second movable shaft member. The slot can have an axial slot portion and a circumferential slot portion located at a distal end of the second movable shaft member. The peg can move in the axial slot portion so that the first movable shaft member can move axially relative to the second movable shaft member. When the sleeve shaft 404 is in the axially extended configuration, rotation of the first movable shaft member relative to the second movable shaft member can cause the peg to move into the circumferential slot portion, thus preventing axial movement of the first movable shaft member relative to the second movable shaft member until the peg is moved back to the axial slot portion.

In some examples, the locking mechanism can include mating threads. For example, a first movable shaft member can have a proximal threaded portion and a second movable shaft member immediately adjacent the first movable shaft member can have a distal threaded portion. When the sleeve shaft 404 is in the axially extended configuration, the proximal threaded portion of the first movable shaft member can engage the distal threaded portion of the second movable shaft member. Thus, rotation of the first movable shaft member relative to the second movable shaft member can threadably engage the first and second movable shaft members, thereby preventing the first movable shaft member from sliding axially relative to the second movable shaft member until the proximal threaded portion is disengaged from the distal threaded portion.

In addition to and/or in lieu of the locking mechanisms described above, other locking mechanisms as known in the art can be included in the sleeve shaft 404.

### Exemplary Fluid Ports

The delivery apparatus 400 can include one or more fluid ports configured to supply flush fluid (e.g., saline solution, heparin solution, etc.) to one or more lumens arranged within the delivery apparatus 400 (e.g., annular lumens arranged between coaxial components of the delivery apparatus 400, etc.) in order to reduce potential thrombus formation.

In the examples depicted in FIGS. 8-12, the delivery apparatus 400 have three fluid ports 450, 452, 454 that are fluidly coupled to respective lumens of the pusher shaft 402, the sleeve shaft 404, and the delivery shaft 406. In some examples, additional fluid ports can be included to flush other portions of the delivery apparatus 400.

For example, the fluid port 450 can be fluidly coupled to the inner lumen 402i of the pusher shaft 402. In certain examples, the fluid port 450 can be located near the proximal end 402p of the pusher shaft 402. In certain examples, a fluid source can be connected to the fluid port 450 to inject a flush fluid into the inner lumen 402i of the pusher shaft 402. The flush fluid can flow through an entire length of the inner lumen 402i and exit at the distal end 402d of the pusher shaft 402, as indicated by the arrows 451. The flow rate of the flush fluid can be adjusted so that it forms a continuous flow within the inner lumen 402i. The inner lumen 402i can be flushed before inserting the delivery apparatus 400 into the patient's vasculature. In addition, a continuous flow of the flush fluid can be maintained within the inner lumen 402i when inserting/navigating the delivery apparatus 400 and deploying the docking device 401.

In some circumstances, e.g., when the distal end 402d of the pusher shaft 402 is spaced away from the docking device 401, the flush fluid exiting at the distal end 402d of the pusher shaft 402 can also flow into a lumen 418i of the dock sleeve 418 and around the docking device 401, and then exit at a distal end of the dock sleeve 418. Thus, the flush fluid introduced through the fluid port 450 can also flush the docking device 401. Notably, the fluid flow (e.g., 451) within the inner lumen 402i of the pusher shaft 402 is one-directional (i.e., in the distal direction) and there is no backflow.

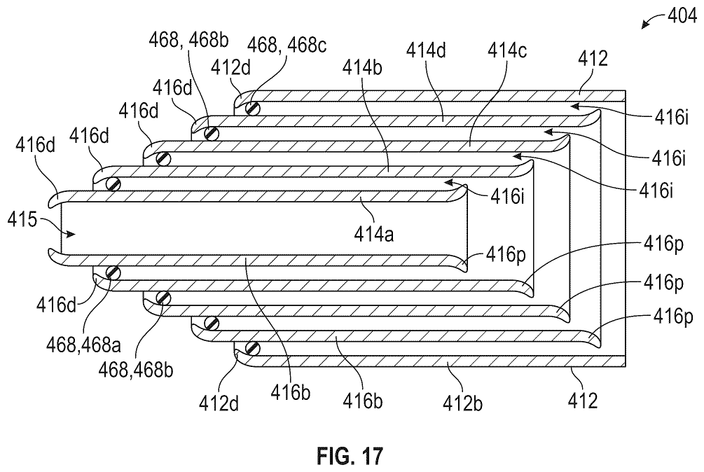

In certain examples, the fluid port 452 can be fluidly coupled to an inner lumen 404i of the sleeve shaft 404. In certain examples, the fluid port 452 can be located near the proximal end 412p of the fixed shaft member 412. Due to the nested structure of the sleeve shaft 404, the inner lumen 404i can comprise the inner lumen 415 of the inner-most shaft member 414a and one or more annular spaces 416i (see, e.g., FIGS. 15C and 17) formed between the movable shaft members 414 and/or the fixed shaft member 412.

In certain examples, a fluid source can be connected to the fluid port 452 to inject a flush fluid into the inner lumen 404i of the sleeve shaft 404. The flush fluid injected through the fluid port 452 can flow through an entire length of the inner lumen 415 of the inner-most shaft member 414a and over the pusher shaft 402. For example, as depicted in FIG. 8, the flush fluid can flow through a flow path or conduit (e.g., an annular space) formed between the pusher shaft 402 and the inner-most shaft member 414a, as indicated by the arrows 453.

Similarly, the flow rate of the flush fluid introduced through the fluid port 452 can be adjusted so that it forms a continuous flow within the inner lumen 415. The inner lumen 415 can be flushed before inserting the delivery apparatus 400 into the patient's vasculature. In addition, a continuous flow of the flush fluid can be maintained within the inner lumen 415 when inserting/navigating the delivery apparatus 400 and deploying the docking device 401.

Because the dock sleeve 418 can be fixedly connected to or an integral part of the inner-most shaft member 414a, the flush fluid injected into the inner lumen 415 can also flow into the lumen 418i of the dock sleeve 418 and around the docking device 401, and then exit at the distal end of the dock sleeve 418. Thus, the flush fluid introduced through the fluid port 452 can also flush the docking device 401. Note that the fluid flow (e.g., 453) within the inner lumen 415 is also one-directional (i.e., in the distal direction) and there is no backflow.

Additionally, the flush fluid injected through the fluid port 452 can also flush the annular spaces 416i (see, e.g., FIGS. 15C and 17) formed between the movable shaft members 414 and/or the fixed shaft member 412. As described further below, the annular spaces 416i can be sealed at respective distal ends such that the flush fluid injected through the fluid port 452 forms a one-dimensional flow (e.g., 453) only within the annular space formed between the pusher shaft 402 and the inner-most shaft member 414a.

In certain examples, the fluid port 454 can be fluidly coupled to the inner lumen 406i of the delivery shaft 406. In certain examples, the fluid port 454 can be located near the proximal end 406p of the delivery shaft 406.

In certain examples, a fluid source can be connected to the fluid port 454 to inject a flush fluid into the inner lumen 406i of the delivery shaft 406. The flush fluid injected through the fluid port 454 can flow through an entire length of the inner lumen 406i of the delivery shaft 406 and over the movable shaft members 414 (if being inserted into the inner lumen 406i) and/or the dock sleeve 418, as indicated by the arrows 455, and exit through a distal end 406d of the delivery shaft 406. Thus, the flush fluid injected through the fluid port 454 can flow unidirectionally (e.g., in the distal direction).

Similarly, the flow rate of the flush fluid introduced through the fluid port 454 can be adjusted so that it forms a continuous flow within the inner lumen 406i. The inner lumen 406i can be flushed before inserting the delivery apparatus 400 into the patient's vasculature. In addition, a continuous flow of the flush fluid can be maintained within the inner lumen 406i when inserting/navigating the delivery apparatus 400 and deploying the docking device 401.

In some examples, the flow rates of the flush fluid introduced into the flush ports 450, 452, and 454 can be independently adjusted so that fluid flows in 402i, 415, and 406i (e.g., as indicated by the arrows 451, 453, and 455) can be the same as or different from each other.

In some examples, the flush ports 450, 452, and 454 can be connected to a common fluid source.

In certain examples, the fluid ports 450, 452, and 454 can be configured to be fluidly “isolated” from each other, meaning that fluid flows indicated by the arrows 451, 453, and 455 are all confined within respective inner lumens (e.g., 402i, 415, and 406i) before existing at respective distal ends (e.g., 402d, 413, 406d). For example, the fluid injected into the fluid port 452 can flow through the conduit formed between the pusher shaft 402 and the inner-most shaft member 414a but do not flow through the inner lumen 402i of the pusher shaft 402. Likewise, the fluid injected into the fluid port 454 can flow through a conduit formed between the delivery shaft 406 and the sleeve shaft 404 and do not flow through the inner lumen 415 of the inner-most shaft member 414a.

Additional details on the handle of a delivery apparatus having one or more flushing ports configured to supply flush fluid to one or more lumens arranged within the delivery apparatus are described in International Patent Applications No. PCT/US2020/36577 and PCT/US2021/059075.

### Exemplary Sealing Mechanisms

In certain examples, the delivery apparatus 400 can include one or more sealing mechanisms configured to prevent leakage of blood, saline, or other fluid through the system.

As an example, the delivery apparatus 400 can include an annular seal 460 configured to seal between the pusher shaft 402 and the delivery shaft 406. In certain cases, the annular seal 460 can be a part of the delivery shaft 406. In certain cases, the annular seal 460 can be located at the proximal end 406p of the delivery shaft 406. Thus, when the sleeve shaft 404 is in the axially collapsed configuration (e.g., no movable shaft member extends into the inner lumen 406i of the delivery shaft 406), the inner-most shaft member 414a can be positioned proximal to the annular seal 460.

In certain examples, the annular seal 460 can comprise a resiliently constricted central opening (e.g., the central opening can be resiliently biased toward a diameter that is smaller than the outer diameter of the inner-most shaft member 414a). As such, the annular seal 460 can resiliently engage with an outer surface of at least one movable shaft member (e.g., 414) when the at least one movable shaft member is inserted into the inner lumen 406i of the delivery shaft 406 (e.g., when the sleeve shaft 404 is in the fully or partially extended configuration). In other words, the diameter of the central opening of the annular seal 460 can adapt to or match different outer diameter of the movable shaft member passing through the annular seal 460, thereby creating a seal between the delivery shaft 406 and the movable shaft member passing through the annular seal. For example, the annular seal 460 can be configured to seal against the outer surface of inner-most shaft member 414a when the inner-most shaft member 414a passes through the annular seal 460, and seal against the outer surface of an intermediate shaft member (e.g., 414b, 414c, 414d, etc.) when the intermediate shaft member passes through the annular seal 460.

In certain examples, the delivery apparatus 400 can also include a sealing member (which can be a part of the sleeve shaft 404) configured to seal one or more annular spaces 416i formed between the one or more movable shaft members 414 and the fixed shaft member 412. As a result, the flush fluid injected through the fluid port 452 can flush but not exit through the annular spaces 416i. FIG. 15C schematically depicts the annular spaces 416i and the inner lumen 415 formed within the sleeve shaft 404.

In certain examples, as shown in FIGS. 15A-15B, the sealing member can include a sealing membrane 462 extending from a distal end portion 412d of the fixed shaft member 412 to the outer surface of the inner-most shaft member 414a, thereby sealing between the fixed shaft member 412 and the outer surface of the inner-most shaft member 414a. In the depicted example, five movable shaft members 414a-414e are shown. A proximal end 462p of the sealing membrane 462 can connect to the outer surface, the inner surface, or the terminal end of the fixed shaft member 412. A distal end of 462d the sealing membrane 462 can connect to the inner-most shaft member 414a at a position adjacent to the distal end 413, or a mid-portion, or a proximal portion of the inner-most shaft member 414a. The sealing membrane 462 can be connected to the fixed shaft member 412 and the inner-most shaft member 414a via any means, such as thermal bonding, gluing, sewing, etc. In certain examples, the sealing membrane 462 can be a single piece surrounding the circumference of the sleeve shaft 404. In other examples, the sealing membrane 462 can include two or more separate pieces, each of which connects the fixed shaft member 412 to the inner-most shaft member 414a.

In certain examples, the sealing membrane 462 can comprise an elastic material (e.g., plastic, rubber, polytetrafluoroethylene (PTFE), polyether block amide (PEBA), etc.) so that the sealing membrane 462 can axially stretch when the inner-most shaft member 414a moves in the distal direction relative to the fixed shaft member 412 (e.g., when the sleeve shaft 404 moves from the axially collapsed configuration to the axially extended configuration). For example, compared to FIG. 15A, FIG. 15B shows the sealing membrane 462 is more axially stretched when the inner-most shaft member 414a is moved to a more distal direction relative to the fixed shaft member 412.

In certain examples, as depicted in FIGS. 16A-16B, the sealing member can include a segmented sealing membrane 464 extending from the distal end portion 412d of the fixed shaft member 412 to the outer surface of the inner-most shaft member 414a. The segmented sealing membrane 464 can be deemed as a special variant of the sealing membrane 462. For example, besides connecting to the fixed shaft member 412 and the inner-most shaft member 414a, the segmented sealing membrane 464 can be further connected to one or more intermediate shaft members (e.g., 414b, 414c, 414d, 414e) so as to divide the segmented sealing membrane 464 into a plurality of sealing segments 466, each of which can be configured to seal a corresponding annular space 416i. In certain examples, the plurality of sealing segments 466 can be connected to respective distal end portion of the intermediate shaft members. Likewise, each of the sealing segments 466 can be a single piece or multiple pieces surrounding a respective shaft member.

In certain examples, the segmented sealing membrane 464 can comprise an elastic material (e.g., similar to 462) so that each sealing segment 466 can axially stretch when the sleeve shaft 404 moves from the axially collapsed configuration to the axially extended configuration). For example, compared to FIG. 16A, FIG. 16B shows the sealing segments 466 are more axially stretched when the inner-most shaft member 414a is moved to a more distal direction relative to the fixed shaft member 412.

In certain examples, the sealing members (e.g., 462, 464) described above can also be configured as an actuation mechanism to cause axial movement of the one or more movable shaft members 414. In one example, bias members can be embedded within or coupled to the sealing members. Additionally, and/or alternatively, bias members can be constructed similar to, but independent of the sealing members. For instance, a bias member (or segmented bias members) can extend from the distal end portion 412d of the fixed shaft member 412 to the outer surface of the inner-most shaft member 414a. In certain examples, the bias member can include tension springs, elastic bands, or the like. As a result, the sleeve shaft 404 in the axially extended configuration can create a tension force in the bias member which urges the sleeve shaft 404 to return to the axially collapsed configuration. Alternatively, the bias member can include compression springs or the like. As a result, the sleeve shaft 404 in the axially collapsed configuration can create a compression force in the bias member which urges the sleeve shaft 404 to return to the axially extended configuration.

In certain examples, as depicted in FIG. 17. The sealing member can include a plurality of annular rings 468 (e.g., O-rings, gaskets, etc.) which are fixedly attached to respective outer surfaces of the one or more movable shaft members 414 and configured to seal respective annular spaces 416i. In the depicted example, four movable shaft members 414a-414d are shown. As shown, one annular ring 468a can be configured to seal between the inner-most shaft member 414a and an intermediate shaft member (e.g., 414b) immediately surrounding the inner-most shaft member 414a. Another annular ring 468c can be configured to seal between the fixed shaft member 412 and an intermediate shaft member (e.g., 414d) immediately surrounded by the fixed shaft member 412. Additional annular rings 468b can be configured to seal between any two intermediate shaft members one of which is immediately surrounded by the other (e.g., between 414b and 414c, between 414c and 414d, etc.).

In certain examples, the sleeve shaft 404 can have a retention mechanism configured to resist movement of the annular rings 468 when the movable shaft members 414 move axially relative to the fixed shaft member 412.

In certain examples, the retention mechanism can comprise crimped distal end portions (e.g., 412d, 416d) of the fixed shaft member 412 and the movable shaft members 414. The crimped distal end portions (e.g., 412d, 416d) can have smaller diameters than respective cylindrical body portions (e.g., 412b, 416b) of the fixed shaft member 412 and the movable shaft members 414. As shown in FIG. 17, the annular rings 468 can be positioned adjacent to the respective crimped distal end portions (e.g., 412d, 416d). As such, the annular rings 468 can be prevented from moving distally past the crimped distal end portions (e.g., 412d, 416d) when the movable shaft members 414 move axially relative to the fixed shaft member 412.

Additionally, or alternatively, retention mechanism can comprise flared proximal end portions 416p of the movable shaft members 414. The flared proximal end portions 416p can have larger diameters than respective cylindrical body portions 416b of the movable shaft members 414. The annular rings 468 can be positioned adjacent to the respective flared proximal end portions 416p. As such, the annular rings 468 can be prevented from moving proximally past the flared proximal end portions 416p when the movable shaft members 414 move axially relative to the fixed shaft member 412.

While specific retention mechanism is depicted in FIG. 17, it is to be understood that the retention mechanism can take other forms. For example, the outer surfaces of the movable shaft members 414 can have annular grooves and the annular rings 468 can be retained in respective grooves. In other examples, the annular rings 468 can be retained in place by other means, such as gluing, fastening, etc.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus configured to deliver a prosthetic implant, the delivery apparatus comprising: a handle; a first shaft extending through the handle; and a second shaft that is coaxial with the first shaft and surrounds at least a proximal end portion of the first shaft, wherein the second shaft comprises an outer shaft member positioned proximal to the handle and an inner shaft member that is axially movable relative to the outer shaft member, wherein the second shaft is movable between an axially extended configuration and an axially collapsed configuration, wherein when the second shaft is in the axially extended configuration, the inner shaft member extends into the handle, wherein when the second shaft is in the axially collapsed configuration, the inner shaft member is positioned proximal to the handle.

Example 2. The delivery apparatus of any example herein, particular example 1, further comprising a suture lock connected to a proximal end of the first shaft, wherein the suture lock is configured to connect to a release suture that is tied to the prosthetic implant.

Example 3. The delivery apparatus of any example herein, particular example 2, wherein a proximal end of the outer shaft member of the second shaft is positioned distal to the proximal end of the first shaft.

Example 4. The delivery apparatus of any example herein, particularly any one of examples 1-3, further comprising a first fluid port that is fluidly coupled to an inner lumen of the first shaft.

Example 5. The delivery apparatus of any example herein, particularly any one of examples 1-4, wherein the second shaft comprises a second fluid port that is fluidly coupled to an annular space formed between the first shaft and the inner shaft member.

Example 6. The delivery apparatus of any example herein, particularly any one of examples 1-5, further comprising a third shaft connected to the handle, wherein the first shaft extends through a lumen of the third shaft.

Example 7. The delivery apparatus of any example herein, particular example 6, wherein the third shaft comprises a third fluid port that is fluidly coupled to the lumen of the third shaft.

Example 8. The delivery apparatus of any example herein, particularly any one of examples 6-7, wherein the third shaft comprises an annular seal configured to seal between the third shaft and the first shaft.

Example 9. The delivery apparatus of any example herein, particular example 8, wherein the annular seal is located at a proximal end of the third shaft, wherein the inner shaft member is positioned proximal to the annular seal when the second shaft is in the axially collapsed configuration.

Example 10. The delivery apparatus of any example herein, particular example 9, wherein the inner shaft member extends into the lumen of the third shaft when the second shaft is in the axially extended configuration.

Example 11. The delivery apparatus of any example herein, particularly any one of examples 9-10, wherein the proximal end of the third shaft extends proximally out of the handle.

Example 12. The delivery apparatus of any example herein, particularly any one of examples 1-11, wherein the second shaft comprises a sealing member configured to seal between the outer shaft member and an outer surface of the inner shaft member.

Example 13. The delivery apparatus of any example herein, particular example 12, wherein the sealing member comprises an elastic sealing membrane extending from a distal end portion of the outer shaft member to the outer surface of the inner shaft member, wherein the elastic sealing membrane is configured to axially stretch when the second shaft moves from the axially collapsed configuration to the axially extended configuration.

Example 14. The delivery apparatus of any example herein, particular example 13, wherein the elastic sealing membrane extends from the distal end portion of the outer shaft member to a distal end portion of the inner shaft member.

Example 15. The delivery apparatus of any example herein, particular example 12, wherein the second shaft comprises one or more intermediate shaft members that are coaxially arranged between the inner shaft member and the outer shaft member.

Example 16. The delivery apparatus of any example herein, particular example 15, wherein the sealing member comprises an elastic sealing membrane extending from a distal end of portion the outer shaft member to the outer surface of the inner shaft member, wherein the elastic sealing membrane is connected to the one or more intermediate shaft members so as to divide the elastic sealing membrane into a plurality of elastic sealing segments, wherein the plurality of elastic sealing segments are configured to axially stretch when the second shaft moves from the axially collapsed configuration to the axially extended configuration.

Example 17. The delivery apparatus of any example herein, particular example 15, wherein the sealing member comprises a plurality of annular rings, wherein each annular ring is configured to seal between the inner shaft member and an intermediate shaft member immediately surrounding the inner shaft member, or between the outer shaft member and an intermediate shaft member immediately surrounded by the outer shaft member, or between two intermediate shaft members one of which is immediately surrounded by the other.

Example 18. The delivery apparatus of any example herein, particular example 17, wherein each of the intermediate shaft members and the outer shaft member has a cylindrical body portion and a crimped distal end portion that has a smaller diameter than the cylindrical body portion, wherein the annular rings are positioned adjacent to respective crimped distal end portions.

Example 19. The delivery apparatus of any example herein, particular example 17, wherein each of the intermediate shaft members and the inner shaft member has a cylindrical body portion and a flared proximal end portion that has a larger diameter than the cylindrical body portion, wherein the annular rings are positioned adjacent to respective flared proximal end portions.

Example 20. The delivery apparatus of any example herein, particularly any one of examples 1-19, further comprising an actuation mechanism configured to move the second shaft between the axially collapsed configuration and the axially extended configuration.

Example 21. The delivery apparatus of any example herein, particular example 20, wherein the actuation mechanism comprises at least one rotational actuator, wherein rotating the at least one rotational actuator in a first direction moves the second shaft from the axially collapsed configuration to the axially extended configuration, and wherein rotating the at least one rotational actuator in a second direction opposite to the first direction moves the second shaft from the axially extended configuration to the axially collapsed configuration.

Example 22. The delivery apparatus of any example herein, particular example 21, wherein the at least one rotational actuator is configured to rotationally engage the second shaft at a location distal to the outer shaft member.

Example 23. The delivery apparatus of any example herein, particularly any one of examples 21-22, wherein the at least one rotational actuator comprises a roller configured to frictionally engage an outer surface of the inner shaft member such that rotational movement of the roller can be converted to axial movement of the inner shaft member.

Example 24. The delivery apparatus of any example herein, particularly any one of examples 21-22, wherein the at least one rotational actuator comprises a nut having internal threads that are configured to engage external threads of the inner shaft member such that rotational movement of the nut can be converted to axial movement of the inner shaft member.

Example 25. The delivery apparatus of any example herein, particularly any one of examples 21-22, wherein the at least one rotational actuator comprises a pinion gear configured to engage with external threads of the inner shaft member such that rotational movement of the pinion gear can be converted to axial movement of the inner shaft member.

Example 26. The delivery apparatus of any example herein, particularly any one of examples 21-22, wherein the at least one rotational actuator comprises a rotatable drive shaft that is hingedly connected to the inner shaft member via a rod such that rotational movement of the drive shaft can be converted to axial movement of the inner shaft member.

Example 27. The delivery apparatus of any example herein, particularly any one of examples 21-22, wherein the at least one rotational actuator comprises a cam that is rotatably connected to the inner shaft member such that rotational movement of the cam can be converted to axial movement of the inner shaft member.

Example 28. The delivery apparatus of any example herein, particular example 20, wherein the actuation mechanism comprises at least one linear actuator, wherein moving the at least one linear actuator in a first direction is configured to move the second shaft from the axially collapsed configuration to the axially extended configuration, and wherein moving the at least one linear actuator in a second direction opposite to the first direction is configured to move the second shaft from the axially extended configuration to the axially collapsed configuration.

Example 29. The delivery apparatus of any example herein, particular example 28, wherein the at least one linear actuator comprises a gripper configured to frictionally engage an outer surface of the inner shaft member when the gripper is radially compressed and detach from the inner shaft member when the gripper is not radially compressed, wherein the gripper is configured to be axially movable relative to the outer shaft member.

Example 30. The delivery apparatus of any example herein, particular example 28, wherein the at least one linear actuator comprises a slider fixedly connected to the inner shaft member, wherein the slider is configured to be axially movable relative to the outer shaft member.

Example 31. The delivery apparatus of any example herein, particular example 20, wherein the actuation mechanism comprises at least one bias member connected to the inner shaft member, wherein the bias member is configured to resiliently bias the inner shaft member axially toward the outer shaft member if the inner shaft member is positioned distally relative to the outer shaft member.

Example 32. The delivery apparatus of any example herein, particular example 20, wherein the actuation mechanism comprises at least one bias member connected to the inner shaft member, wherein the bias member is configured to resiliently bias the inner shaft member axially away from the outer shaft member if the inner shaft member is positioned within the outer shaft member.

Example 33. A delivery apparatus configured to deliver a prosthetic implant, the delivery apparatus comprising: a first shaft comprising a lumen and a first fluid port that is fluidly coupled to the lumen of the first shaft; and a second telescopic shaft comprising an outer shaft member fixedly mounted around a proximal portion of the first shaft and one or more inner shaft members that are coaxial with and axially movable relative to the first shaft, wherein the one or more inner shaft members comprise an inner-most shaft member, wherein the first shaft extends through a lumen of the inner-most shaft member, wherein the second telescopic shaft comprises a second fluid port that is fluidly coupled to the lumen of the inner-most shaft member.

Example 34. The delivery apparatus of any example herein, particular example 33, wherein a fluid injected into the second fluid port flows through a conduit formed between the first shaft and the inner-most shaft member and does not flow through the lumen of the first shaft.

Example 35. The delivery apparatus of any example herein, particularly any one of examples 33-34, further comprising a handle, wherein the first shaft extends longitudinally through the handle, wherein the outer shaft member is positioned proximal relative to the handle.

Example 36. The delivery apparatus of any example herein, particular example 35, wherein at least some of the one or more inner shaft members are configured to be axially extendable into the handle.

Example 37. The delivery apparatus of any example herein, particularly any one of examples 33-36, further comprising a dock sleeve connected to a distal end of the inner-most shaft member, wherein the dock sleeve is configured to cover the prosthetic implant.

Example 38. The delivery apparatus of any example herein, particularly any one of examples 33-37, further comprising a suture lock connected to a proximal end of the first shaft, wherein the suture lock is configured to be releasably connected to the prosthetic implant via a release suture extending through the lumen of the first shaft.

Example 39. The delivery apparatus of any example herein, particularly any one of examples 33-38, further comprising a third shaft positioned distal to the outer shaft member, wherein the first shaft extends through a lumen of the third shaft.

Example 40. The delivery apparatus of any example herein, particular example 39, wherein the third shaft comprises a third fluid port that is fluidly coupled to the lumen of the third shaft.

Example 41. The delivery apparatus of any example herein, particular example 40, wherein a fluid injected into the third fluid port flows through a conduit formed between the second telescopic shaft and the third shaft and does not flow through the lumen of the inner-most shaft member.

Example 42. The delivery apparatus of any example herein, particularly any one of examples 39-41, wherein at least some of the one or more inner shaft members are configured to be axially extendable into the lumen of the third shaft.

Example 43. The delivery apparatus of any example herein, particularly any one of examples 39-42, further comprising an annular seal configured to seal between the first shaft and the third shaft.

Example 44. The delivery apparatus of any example herein, particular example 43, wherein the annular seal comprises a resiliently constricted central opening so as to resiliently engage with an outer surface of at least one inner shaft member when the at least one inner shaft member is inserted into the lumen of the third shaft, thereby creating a seal between the third shaft and the at least one inner shaft member.

Example 45. The delivery apparatus of any example herein, particularly any one of examples 33-44, further comprising a sealing member configured to seal between the outer shaft member and an outer surface of the inner-most shaft member.

Example 46. The delivery apparatus of any example herein, particular example 45, wherein the sealing member comprises an elastic sealing membrane extending from a distal end portion of the outer shaft member to the outer surface of the inner-most shaft member, wherein the elastic sealing membrane is configured to axially stretch when the inner-most shaft member moves in a distal direction relative to the outer shaft member.

Example 47. The delivery apparatus of any example herein, particular example 46, wherein the elastic sealing membrane comprises one or more elastic sealing segments that are respectively connected to the one or more inner shaft members.

Example 48. The delivery apparatus of any example herein, particularly any one of examples 45-46, wherein the sealing member comprises one or more annular rings configured to seal respective annular spaces formed between the one or more inner shaft members and the outer shaft member.

Example 49. The delivery apparatus of any example herein, particularly any one of examples 33-48, wherein each of the one or more inner shaft members comprise a lubricant coating.

Example 50. The delivery apparatus of any example herein, particularly any one of examples 33-49, further comprising an actuation mechanism configured to successively move the one or more inner shaft members in a distal direction toward a distal end of the first shaft or successively retract the one or more inner shaft members in a proximal direction toward the outer shaft member.

Example 51. A delivery apparatus configured to deliver a prosthetic implant, the delivery apparatus comprising: a first shaft; a second shaft comprising an outer shaft member fixedly mounted around a proximal portion of the first shaft and one or more inner shaft members that are nested within one another and configured to be axially movable relative to one another; and a sealing member configured to seal one or more annular spaces formed between the one or more inner shaft members and the outer shaft member, wherein the one or more inner shaft members comprise an inner-most shaft member, wherein the first shaft extends through the inner-most shaft member.

Example 52. The delivery apparatus of any example herein, particular example 51, further comprising a handle, wherein the first shaft extends longitudinally through the handle.

Example 53. The delivery apparatus of any example herein, particular example 52, wherein the outer shaft member is positioned proximal relative to the handle, wherein at least one of the one or more inner shaft members is configured to be axially extendable into the handle.

Example 54. The delivery apparatus of any example herein, particularly any one of examples 51-53, further comprising a suture lock connected to a proximal end of the first shaft, wherein the suture lock is configured to be releasably connected to the prosthetic implant via a release suture extending through the first shaft.

Example 55. The delivery apparatus of any example herein, particularly any one of examples 51-54, further comprising a first fluid port that is fluidly coupled to a lumen of the first shaft.

Example 56. The delivery apparatus of any example herein, particularly any one of examples 51-55, further comprising a second fluid port that is fluidly coupled to a lumen of the inner-most shaft member.

Example 57. The delivery apparatus of any example herein, particularly any one of examples 51-56, further comprising a third shaft positioned distal to the outer shaft member, wherein the first shaft extends through a lumen of the third shaft.

Example 58. The delivery apparatus of any example herein, particular example 57, further comprising a third fluid port that is fluidly coupled to the lumen of the third shaft.

Example 59. The delivery apparatus of any example herein, particularly any one of examples 57-58, further comprising an annular seal configured to seal between the third shaft and the first shaft when no inner shaft member is inserted into the lumen of the third shaft, or seal between the third shaft and an inner shaft member when the inner shaft member is inserted into the lumen of the third shaft.

Example 60. The delivery apparatus of any example herein, particularly any one of examples 51-59, wherein the second shaft is movable between an axially extended configuration and an axially collapsed configuration, wherein the second shaft has a larger axial length in the axially extended configuration than in the axially collapsed configuration.

Example 61. The delivery apparatus of any example herein, particular example 60, wherein in the axially collapsed configuration, each of the one or more inner shaft members is substantially received within the outer shaft member, wherein in the axially extended configuration, each of the one or more inner shaft members substantially extends out of the outer shaft member.

Example 62. The delivery apparatus of any example herein, particular example 61, wherein in the axially extended configuration, the one or more inner shaft members are configured such that an inner shaft member having a smaller diameter extends to a more distal position than an inner shaft member having a larger diameter.

Example 63. The delivery apparatus of any example herein, particularly any one of examples 51-62, wherein the sealing member comprises an elastic sealing membrane extending from a distal end portion of the outer shaft member to an outer surface of the inner-most shaft member, wherein the elastic sealing membrane is configured to axially stretch when the inner-most shaft member moves in a distal direction relative to the outer shaft member.

Example 64. The delivery apparatus of any example herein, particular example 63, wherein the elastic sealing membrane is connected to the one or more inner shaft members so as to define one or more elastic sealing segments, wherein each elastic sealing segment is configured to seal a corresponding annular space.

Example 65. The delivery apparatus of any example herein, particularly any one of examples 51-64, wherein the sealing member comprises one or more annular rings configured to seal respective annular spaces formed between the one or more inner shaft members and the outer shaft member.

Example 66. The delivery apparatus of any example herein, particular example 65, wherein the second shaft comprises a retention mechanism configured to resist movement of the annular rings when the inner shaft members move axially relative to the outer shaft member.

Example 67. The delivery apparatus of any example herein, particular example 66,wherein the retention mechanism comprises crimped distal end portions of the outer shaft member and the one or more inner shaft members, wherein the crimped distal end portions have smaller diameters than respective body portions of the outer shaft member and the one or more inner shaft members.

Example 68. The delivery apparatus of any example herein, particular example 66, wherein the retention mechanism comprises flared proximal end portions of the one or more inner shaft members, wherein the flared proximal end portions have larger diameters than respective body portions of the one or more inner shaft members.

Example 69. The delivery apparatus of any example herein, particularly any one of examples 51-68, further comprising an actuation mechanism configured to move the one or more inner shaft members out of the outer shaft member so that distal ends of the one or more inner shaft members are moved to progressively more distal positions along the first shaft, and wherein the actuation mechanism is further configured to move the one or more inner shaft members substantially into the outer shaft member.

Example 70. The delivery apparatus of any example herein, particular example 69, wherein the actuation mechanism comprises a motor operatively coupled to the one or more inner shaft members.

Example 71. A system, comprising: a prosthetic implant; and a delivery apparatus configured to deliver the prosthetic implant to a target implantation location, the delivery apparatus comprising: a pusher shaft positioned proximal to the prosthetic implant; a sleeve shaft comprising an outer shaft member fixedly mounted around a proximal portion of the pusher shaft and one or more inner shaft members that are coaxial with and axially movable relative to the pusher shaft; and a suture lock connected to a proximal end of the pusher shaft, wherein the suture lock is releasably connected to the prosthetic implant via a release suture extending through the pusher shaft, wherein the one or more inner shaft members comprise an inner-most shaft member, wherein the pusher shaft extends through the inner-most shaft member.

Example 72. The system of any example herein, particular example 71, wherein the prosthetic implant is a prosthetic valve.

Example 73. The system of any example herein, particular example 71, wherein the prosthetic implant is a docking device configured to receive a prosthetic valve.

Example 74. The system of any example herein, particularly any one of examples 71-73, wherein the delivery apparatus further comprises a first fluid port that is fluidly coupled to a lumen of the pusher shaft.

Example 75. The system of any example herein, particularly any one of examples 71-74, wherein the delivery apparatus further comprises a second fluid port that is fluidly coupled to a lumen of the inner-most shaft member.

Example 76. The system of any example herein, particularly any one of examples 71-75, wherein the delivery apparatus further comprises a handle positioned distal to the outer shaft member, wherein the pusher shaft extends longitudinally through the handle.

Example 77. The system of any example herein, particular example 76, wherein the delivery apparatus further comprises a delivery shaft connected to the handle, wherein the pusher shaft extends through a lumen of the delivery shaft.

Example 78. The system of any example herein, particular example 77, wherein the delivery shaft is positioned distal to the outer shaft member, wherein at least some of the one or more inner shaft members are configured to be axially extendable into the lumen of the delivery shaft.

Example 79. The system of any example herein, particularly any one of examples 77-78, a third fluid port that is fluidly coupled to the lumen of the delivery shaft.

Example 80. The system of any example herein, particularly any one of examples 77-79, wherein the delivery apparatus further comprises an annular seal configured to seal between the pusher shaft and the delivery shaft when no inner shaft member extends into the lumen of the delivery shaft.

Example 81. The system of any example herein, particular example 80, wherein the annular seal is configured to seal between the delivery shaft and an inner shaft member when the inner shaft member extends into the lumen of the delivery shaft.

Example 82. The system of any example herein, particularly any one of examples 71-81, wherein the delivery apparatus further comprises a sealing member configured to seal one or more annular spaces formed between the one or more inner shaft members and the outer shaft member.

Example 83. The system of any example herein, particular example 82, wherein the sealing member comprises an elastic sealing membrane extending from a distal end portion of the outer shaft member to an outer surface of the inner-most shaft member, wherein the elastic sealing membrane is configured to axially stretch when the inner-most shaft member moves in a distal direction relative to the outer shaft member.

Example 84. The system of any example herein, particular example 83, wherein the elastic sealing membrane comprises one or more elastic sealing segments respectively connected to the one or more inner shaft members, wherein each elastic sealing segment is configured to seal a corresponding annular space.

Example 85. The system of any example herein, particularly any one of examples 82-84, wherein the sealing member comprises one or more annular rings configured to seal respective annular spaces.

Example 86. The system of any example herein, particular example 85, wherein the one or more annular rings are fixedly attached to respective outer surfaces of the one or more inner shaft members.

Example 87. The system of any example herein, particularly any one of examples 71-86, wherein the delivery apparatus further comprises an actuation mechanism configured to telescopically move the one or more inner shaft members in a distal direction relative to the outer shaft member or telescopically move the one or more inner shaft members in a proximal direction toward the outer shaft member.

Example 88. The system of any example herein, particular example 87, wherein the actuation mechanism comprises at least one rotational actuator operatively connected to the one or more inner shaft members, wherein a rotation of the at least one rotational actuator is configured to telescopically move the one or more inner shaft members axially relative to the outer shaft member.

Example 89. The system of any example herein, particular example 87, wherein the actuation mechanism comprises at least one linear actuator operatively connected to the one or more inner shaft members, wherein a linear translation of the linear actuator is configured to telescopically move the one or more inner shaft members axially relative to the outer shaft member.

Example 90. The system of any example herein, particular example 87, wherein the actuation mechanism comprises at least one bias member operatively connected to the one or more inner shaft members, wherein the bias member is configured to telescopically move the one or more inner shaft members axially relative to the outer shaft member when positions of the one or more inner shaft members relative to the outer shaft member create a biasing force that moves the bias member from a biased state to an unbiased state.

Example 91. A method, comprising: inserting a delivery apparatus loaded with a prosthetic device into a vasculature of a patient; and deploying the prosthetic device at a target location within the vasculature of the patient, wherein the delivery apparatus comprises: a pusher shaft positioned proximal to the prosthetic device; a sleeve shaft comprising an outer shaft member fixedly mounted around a proximal portion of the pusher shaft and one or more inner shaft members that are coaxial with and axially movable relative to the pusher shaft; and a delivery shaft surrounding at least a distal portion of the pusher shaft and positioned distal to the outer shaft member, wherein the one or more inner shaft members comprise an inner-most shaft member, wherein the pusher shaft extends through the inner-most shaft member.

Example 92. The method of any example herein, particular example 91, wherein deploying the prosthetic device comprises cutting a release suture tied to the prosthetic device, wherein the release suture extends through the pusher shaft and is connected to a suture lock connected to a proximal end of the pusher shaft.

Example 93. The method of any one of any example herein, particular example 91-92, further comprising flushing a lumen of the pusher shaft with a solution.

Example 94. The method of any example herein, particular example 93, wherein flushing the lumen of the pusher shaft comprises injecting the solution from a first fluid port that is fluidly coupled to the lumen of the pusher shaft.

Example 95. The method of any example herein, particularly any one of examples 93-94, further comprising maintaining a continuous flow of the solution within the lumen of the pusher shaft when inserting the delivery apparatus and deploying the prosthetic device.

Example 96. The method of any example herein, particularly any one of examples 93-96, further comprising flushing an annular space formed between the inner-most shaft member and the pusher shaft with the solution.

Example 97. The method of any example herein, particular example 96, wherein flushing the annular space between the inner-most shaft member and the pusher shaft comprises injecting the solution from a second fluid port that is fluidly coupled to a lumen of the inner-most shaft member.

Example 98. The method of any example herein, particularly any one of examples 93-97, further comprising flushing an annular space formed between the delivery shaft and the pusher shaft with the solution.

Example 99. The method of any example herein, particular example 98, wherein flushing the annular space formed between the delivery shaft and the pusher shaft comprises injecting the solution from a third fluid port that is fluidly coupled to a lumen of the delivery shaft.

Example 100. The method of any example herein, particularly any one of examples 91-99, wherein inserting the delivery apparatus comprises selectively flexing a distal portion of the delivery shaft.

Example 101. The method of any example herein, particularly any one of examples 91-100, further comprising moving the one or more inner shaft members in a distal direction relative to the outer shaft member so that a dock sleeve covers the prosthetic device when inserting the delivery apparatus, wherein the dock sleeve is connected to a distal end of the inner-most shaft member.

Example 102. The method of any example herein, particular example 101, wherein deploying the prosthetic device comprises moving the prosthetic device out of a distal end of the delivery shaft while keeping the prosthetic device being covered by the dock sleeve.

Example 103. The method of any example herein, particular example 102, further comprising moving the one or more inner shaft members in a proximal direction relative to the outer shaft member so that the dock sleeve is removed from the prosthetic device.

Example 104. The method of any example herein, particularly any one of examples 101-103, wherein moving the one or more inner shaft members comprises actuating an actuation mechanism operatively coupled to the one or more inner shaft members.

Example 105. The method of any example herein, particular example 104, wherein actuating the actuation mechanism comprises rotating at least one rotational actuator, wherein rotating the at least one rotational actuator in a first direction causes the inner-most shaft member to move distally and cover the prosthetic device, and wherein rotating the at least one rotational actuator in a second direction opposite to the first direction causes the inner-most shaft member to move proximally and uncover the prosthetic device.

Example 106. The method of any example herein, particular example 104, wherein actuating the actuation mechanism comprises translating at least one linear actuator, wherein translating the at least one linear actuator in a first direction causes the inner-most shaft member to move distally and cover the prosthetic device, and wherein translating the at least one linear actuator in a second direction opposite to the first direction causes the inner-most shaft member to move proximally and uncover the prosthetic device.

Example 107. The method of any example herein, particular example 104, wherein actuating the actuation mechanism comprises moving a bias member connected to the inner-most shaft member between a biased state and an unbiased state, wherein the bias member is in the biased state when the dock sleeve covers the prosthetic device, and wherein the bias member is configured to return to the unbiased state when the dock sleeve uncovers the prosthetic device.

Example 108. A delivery apparatus configured to deliver a prosthetic implant, the delivery apparatus comprising: a pusher shaft; a telescopic sleeve shaft comprising a fixed shaft segment and one or more movable shaft segments that are coaxial with the fixed shaft segment; and a dock sleeve connected to one of the one or more movable shaft segments and configured to cover the prosthetic implant, wherein the fixed shaft segment is fixedly mounted around a proximal portion of the pusher shaft, wherein the one or more movable shaft segments are axially movable relative to the pusher shaft.

Example 109. The delivery apparatus of any example herein, particular example 108, wherein the fixed shaft segment has a larger diameter than the one or more movable shaft segments.

Example 110. The delivery apparatus of any example herein, particular example 109, wherein the one or more movable shaft segments comprise an inner-most shaft segment, wherein the pusher shaft extends through a lumen of the inner-most shaft segment, wherein the dock sleeve is connected to a distal end of the inner-most shaft segment.

Example 111. The delivery apparatus of any example herein, particular example 108, wherein the fixed shaft segment has a smaller diameter than the one or more movable shaft segments, wherein the pusher shaft extends through a lumen of the fixed shaft segment.

Example 112. The delivery apparatus of any example herein, particular example 111, wherein the one or more movable shaft segments comprise an outer-most shaft segment, wherein the dock sleeve is connected a distal end of the outer-most shaft segment.

Example 113. The delivery apparatus of any example herein, particularly any one of examples 108-112, further comprising a sealing member configured to seal annular spaces formed between the fixed shaft segment and the one or more movable shaft segments.

Example 114. The delivery apparatus of any example herein, particularly any one of examples 108-113, further comprising a first fluid port fluidly coupled to a lumen of the pusher shaft and a second fluid port fluidly coupled to an annular space between the pusher shaft and the telescopic sleeve shaft.

Example 115. The delivery apparatus of any example herein, particularly any one of examples 108-114, further comprising an actuation mechanism configured to axially move the one or more movable shaft segments relative to the fixed shaft segment.

Example 116. A method comprising sterilizing apparatus, devices, assemblies, and/or systems of any examples herein, particularly any one of examples 1-90 and 108-115.

The features described herein with regard to any example can be combined with other features described in any one or more of the other examples, unless otherwise stated. For example, any one or more of the features of one delivery apparatus can be combined with any one or more features of another delivery apparatus.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A delivery apparatus configured to deliver a prosthetic implant, the delivery apparatus comprising:
a handle;
a first shaft extending through the handle; and
a second shaft that is coaxial with the first shaft and surrounds at least a proximal end portion of the first shaft,
wherein the second shaft comprises an outer shaft member positioned proximal to the handle and an inner shaft member that is axially movable relative to the outer shaft member,
wherein the second shaft is movable between an axially extended configuration and an axially collapsed configuration,
wherein when the second shaft is in the axially extended configuration, the inner shaft member extends into the handle,
wherein when the second shaft is in the axially collapsed configuration, the inner shaft member is positioned proximal to the handle.

2. The delivery apparatus of claim 1, further comprising a suture lock connected to a proximal end of the first shaft, wherein the suture lock is configured to connect to a release suture that is tied to the prosthetic implant.

3. The delivery apparatus of claim 2, wherein a proximal end of the outer shaft member of the second shaft is positioned distal to the proximal end of the first shaft.

4. The delivery apparatus of claim 1, further comprising a first fluid port that is fluidly coupled to an inner lumen of the first shaft.

5. The delivery apparatus of claim 1, wherein the second shaft comprises a second fluid port that is fluidly coupled to an annular space formed between the first shaft and the inner shaft member.

6. The delivery apparatus of claim 1, further comprising a third shaft connected to the handle, wherein the first shaft extends through a lumen of the third shaft.

7. The delivery apparatus of claim 6, wherein the third shaft comprises a third fluid port that is fluidly coupled to the lumen of the third shaft.

8. The delivery apparatus of claim 6, wherein the third shaft comprises an annular seal configured to seal between the third shaft and the first shaft.

9. The delivery apparatus of claim 8, wherein the annular seal is located at a proximal end of the third shaft, wherein the inner shaft member is positioned proximal to the annular seal when the second shaft is in the axially collapsed configuration.

10. The delivery apparatus of claim 9, wherein the inner shaft member extends into the lumen of the third shaft when the second shaft is in the axially extended configuration.

11. The delivery apparatus of claim 9, wherein the proximal end of the third shaft extends proximally out of the handle.

12. The delivery apparatus of claim 1, wherein the second shaft comprises a sealing member configured to seal between the outer shaft member and an outer surface of the inner shaft member.

13. The delivery apparatus of claim 12, wherein the sealing member comprises an elastic sealing membrane extending from a distal end portion of the outer shaft member to the outer surface of the inner shaft member, wherein the elastic sealing membrane is configured to axially stretch when the second shaft moves from the axially collapsed configuration to the axially extended configuration.

14. A delivery apparatus configured to deliver a prosthetic implant, the delivery apparatus comprising:
a first shaft comprising a lumen and a first fluid port that is fluidly coupled to the lumen of the first shaft; and
a second telescopic shaft comprising an outer shaft member fixedly mounted around a proximal portion of the first shaft and one or more inner shaft members that are coaxial with and axially movable relative to the first shaft,
wherein the one or more inner shaft members comprise an inner-most shaft member, wherein the first shaft extends through a lumen of the inner-most shaft member,
wherein the second telescopic shaft comprises a second fluid port that is fluidly coupled to the lumen of the inner-most shaft member.

15. The delivery apparatus of claim 14, wherein a fluid injected into the second fluid port flows through a conduit formed between the first shaft and the inner-most shaft member and does not flow through the lumen of the first shaft.

16. The delivery apparatus of claim 14, further comprising a handle, wherein the first shaft extends longitudinally through the handle, wherein the outer shaft member is positioned proximal relative to the handle.

17. The delivery apparatus of claim 16, wherein at least some of the one or more inner shaft members are configured to be axially extendable into the handle.

18. A delivery apparatus configured to deliver a prosthetic implant, the delivery apparatus comprising:
a first shaft;
a second shaft comprising an outer shaft member fixedly mounted around a proximal portion of the first shaft and one or more inner shaft members that are nested within one another and configured to be axially movable relative to one another; and
a sealing member configured to seal one or more annular spaces formed between the one or more inner shaft members and the outer shaft member,
wherein the one or more inner shaft members comprise an inner-most shaft member, wherein the first shaft extends through the inner-most shaft member.

19. The delivery apparatus of claim 18, further comprising a handle, wherein the first shaft extends longitudinally through the handle.

20. The delivery apparatus of claim 19, wherein the outer shaft member is positioned proximal relative to the handle, wherein at least one of the one or more inner shaft members is configured to be axially extendable into the handle.
